SEQUENCE LISTING (14 sequences)

SEQ ID NO: 1 - protein, 1314 aa, Clostridium tetani
PITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 2 - DNA, 6816 bp, Artificial sequence
  vector pRSET-TeNT encoding TeNT
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccgagctcgagccgattaccatcaataattttcgttattcagatccggtgaataatgataccatcatcatgatggaaccaccgtactgtaaagggctggatatttattataaagcgttcaaaatcaccgaccgcatctggatcgtgccggaacgctacgaattcggcaccaaaccggaagattttaatccgccgagtagtctgatcgaaggtgcatcggaatactacgatccgaattatctgcgtactgactctgataaagatcgctttctgcaaacgatggtaaaactgttcaatcgtatcaaaaacaatgtagcaggcgaagccctgctggataaaatcatcaacgccattccgtatctgggaaacagttattctctgctggataaattcgatacaaactcgaactctgtgtcattcaacctgctggaacaggacccgagcggcgcgaccactaagagtgcgatgctgactaacctgattattttcggtccgggaccggtactgaataaaaatgaagttcgcggcattgtactgcgtgtcgataataaaaactatttcccatgtcgtgatggcttcggcagcatcatgcagatggccttttgtccggaatatgtgccaactttcgataatgtgattgagaacatcacctctctgacgattggtaaaagtaaatatttccaggatccggctctgctgctgatgcatgaactgatccatgttctgcatggcctgtatggcatgcaggtttcatcccacgaaattatcccatccaaacaggaaatttacatgcagcatacatatccgattagtgccgaagaactgttcacttttggcggccaggatgcgaacctgatttcgattgacattaaaaacgatctgtatgaaaaaactctgaacgattataaagcgattgccaacaaactgtctcaggtaacctcctgtaacgatccgaatattgatattgacagttataaacaaatttatcagcagaagtatcagttcgataaagactctaatggccagtatattgttaacgaagataaattccagattctgtacaatagcattatgtatggctttactgagatcgaactgggtaaaaaatttaacatcaagactcgtctgagctattttagcatgaatcatgatccagtgaaaatcccgaatctgctggatgatacgatttataatgataccgaaggatttaacatcgaaagcaaggatctgaaatccgaatataaagggcagaacatgcgcgttaataccaatgcatttcgcaatgttgatggttcaggcctggtgtcgaaactgattgggctgtgtaagaaaatcattccaccgacaaatattcgcgaaaatctgtacaaccgtacggcgagcctgaccgatctggggggagaactgtgtattaaaatcaaaaatgaagatctgaccttcattgctgagaagaatagcttttccgaagagccattccaggacgaaatcgtgtcttataacaccaagaataaaccgctgaatttcaactactccctggacaaaatcattgtggattacaacctgcagagtaaaattaccctgccgaatgatcgtaccaccccggtgacgaaaggcatcccttacgcaccagaatataaatcaaatgcagcctcgactatcgagatccataatattgatgacaacactatttaccagtacctgtatgctcagaaatctccgacgacgctgcagcgcatcaccatgactaacagcgtggacgatgccctgattaatagcaccaaaatctactcttactttccgtcggtgatctctaaggttaatcagggcgcgcaaggtatcctgtttctgcaatgggtgcgtgatattattgatgatttcactaatgaatctagccagaaaacgacaattgataaaatttcggatgtttccaccatcgtgccttacatcggcccagcgctgaacatcgtgaagcagggttatgagggtaactttatcggagcactggaaacgaccggcgtggttctgctgctggaatatattccggagattactctgccagttattgcggctctgtcgattgcagagagctcaacgcagaaagaaaaaattattaagacgatcgacaatttcctggaaaagcgctacgaaaaatggatcgaagtgtataagctggtgaaagcgaaatggctggggaccgtgaacacccagttccaaaaacgttcctatcaaatgtatcgtagcctggaatatcaggtggacgccattaaaaagatcatcgattacgaatataagatctactccggtccggacaaagaacagattgcggacgaaattaacaatctgaaaaataaactggaggaaaaagccaacaaagcgatgattaatatcaatattttcatgcgtgaaagcagccgtagcttcctggtcaatcagatgattaatgaagcgaagaaacaactgctggaatttgatacgcaatctaaaaatattctgatgcaatacatcaaagccaattctaaatttattgggatcacggaactgaaaaagctggaatcgaaaatcaataaagtctttagcaccccgattccgttctcctactcgaaaaatctggattgttgggttgacaatgaagaagatattgatgttattctgaaaaagagcacgatcctgaacctggatattaataacgatattatctctgatatcagtggttttaattcatcagttattacttacccagacgctcaactggtgccgggaatcaatgggaaagccattcatctggtgaataatgaatcaagtgaagtgatcgtgcataaagcgatggatatcgagtacaacgatatgtttaataatttcacggtgtcgttctggctgcgtgttccgaaagtgagtgcctcccacctggaacaatatggaaccaacgaatactcaatcattagcagcatgaagaaacattcgctgagtattggttcaggttggagcgtttccctgaaagggaacaatctgatctggacactgaaggactcagcgggcgaagtgcgtcagattacgtttcgtgatctgccggataaatttaatgcatacctggctaacaaatgggtgttcatcacaatcaccaatgaccgtctgtcgtctgcaaacctgtatattaatggggtactgatgggctcggcagaaattacagggctgggcgccattcgtgaagataacaatattacgctgaaactggatcgttgtaataacaataatcagtatgtgagcattgataaatttcgtattttctgcaaagcgctgaacccgaaagaaattgaaaaactgtatacctcgtatctgtcaattacgtttctgcgcgatttctggggaaacccgctgcgttacgatacggaatactacctgatcccggtagccagttctagtaaagacgttcaactgaaaaatattaccgactacatgtatctgacaaacgctccatcatacacaaacggcaaactgaacatctattaccgtcgcctgtacaatgggctgaaattcatcattaaacgttataccccgaataacgaaattgattcctttgtgaagtccggtgacttcattaagctgtatgtatcctataacaataatgaacacatcgttggctatccgaaggatggcaatgcctttaacaacctggatcgtattctgcgtgtaggttacaacgccccgggtattccgctgtataagaaaatggaagcagtgaaactgcgtgatctgaaaacatattccgtgcaactgaagctgtatgatgacaaaaatgctagcctgggtctggtaggcacgcataacggtcagattggaaacgatcctaatcgtgacatcctgatcgcctctaactggtattttaaccacctgaaagataaaattctgggctgcgattggtattttgtccctaccgatgaaggctggacgaacgattaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 3 - protein, 858 aa, Clostridium tetani
SLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 4 - protein, 451 aa, Clostridium tetani
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 5 - protein, 1314 aa, Clostridium tetani
PITINNFRYSDPVNNDTIIMMEPPYCKGLDIYYKAFKITDRIWIVPERYEFGTKPEDFNPPSSLIEGASEYYDPNYLRTDCDKDRFLQTMVKLFNRIKNNVAGEALLDKIINAIPYLGNCYSLLDKFDTNSNSVSFNLLEQDPCGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSCHEIIPSKQEIYMQHTYPISAEELFTFGGQDANLISIDIKNDLYEKTLNDYKAIANKLSQVTSCNDPNIDIDSYKQIYQQKYQFDKDCNGQYIVNEDKFQILYNSIMYGFTEIELGKKFNIKTRLSYFSMNHDPVKIPNLLDDTIYNDTEGFNIESKDLKSEYKGQNMRVNTNAFRNVDGCGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVICKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSACHLEQYGTNEYSIISSMKKHSLSIGSGWSVSLKGNNLIWTLKDSAGEVRQITFRDLPDKFNAYLANKWVFITITNDRLCSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPCYTNGKLNIYYRRLYNGLKFIIKRYTPNNEIDCFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 6 - DNA, 6816 bp, Artificial sequence
  vector pRSET-TeNT encoding the surface serine to cysteine substituted mature TeNT of SEQ ID NO 5
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccgagctcgagccgatcaccatcaacaacttccgttactctgacccggttaacaacgacaccatcatcatgatggaaccgccgtactgcaaaggtctggacatctactacaaagcgttcaaaatcaccgaccgtatctggatcgttccggaacgttacgaattcggtaccaaaccggaagacttcaacccgccgtcttctctgatcgaaggtgcgtctgaatactacgacccgaactacctgcgtaccgactgcgacaaagaccgtttcctgcagaccatggttaaactgttcaaccgtatcaaaaacaacgttgcgggtgaagcgctgctggacaaaatcatcaacgcgatcccgtacctgggtaactgctactctctgctggacaaattcgacaccaactctaactctgtttctttcaacctgctggaacaggacccgtgcggtgcgaccaccaaatctgcgatgctgaccaacctgatcatcttcggtccgggtccggttctgaacaaaaacgaagttcgtggtatcgttctgcgtgttgacaacaaaaactacttcccgtgccgtgacggtttcggttctatcatgcagatggcgttctgcccggaatacgttccgaccttcgacaacgttatcgaaaacatcacctctctgaccatcggtaaatctaaatacttccaggacccggcgctgctgctgatgcacgaactgatccacgttctgcacggtctgtacggtatgcaggtttcttgccacgaaatcatcccgtctaaacaggaaatctacatgcagcacacctacccgatctctgcggaagaactgttcaccttcggtggtcaggacgcgaacctgatctctatcgacatcaaaaacgacctgtacgaaaaaaccctgaacgactacaaagcgatcgcgaacaaactgtctcaggttacctcttgcaacgacccgaacatcgacatcgactcttacaaacagatctaccagcagaaataccagttcgacaaagactgcaacggtcagtacatcgttaacgaagacaaattccagatcctgtacaactctatcatgtacggtttcaccgaaatcgaactgggtaaaaaattcaacatcaaaacccgtctgtcttacttctctatgaaccacgacccggttaaaatcccgaacctgctggacgacaccatctacaacgacaccgaaggtttcaacatcgaatctaaagacctgaaatctgaatacaaaggtcagaacatgcgtgttaacaccaacgcgttccgtaacgttgacggttgcggtctggtttctaaactgatcggtctgtgcaaaaaaatcatcccgccgaccaacatccgtgaaaacctgtacaaccgtaccgcgtctctgaccgacctgggtggtgaactgtgcatcaaaatcaaaaacgaagacctgaccttcatcgcggaaaaaaactctttctctgaagaaccgttccaggacgaaatcgtttcttacaacaccaaaaacaaaccgctgaacttcaactactctctggacaaaatcatcgttgactacaacctgcagtctaaaatcaccctgccgaacgaccgtaccaccccggttaccaaaggtatcccgtacgcgccggaatacaaatctaacgcggcgtctaccatcgaaatccacaacatcgacgacaacaccatctaccagtacctgtacgcgcagaaatctccgaccaccctgcagcgtatcaccatgaccaactctgttgacgacgcgctgatcaactctaccaaaatctactcttacttcccgtctgttatctgcaaagttaaccagggtgcgcagggtatcctgttcctgcagtgggttcgtgacatcatcgacgacttcaccaacgaatcttctcagaaaaccaccatcgacaaaatctctgacgtttctaccatcgttccgtacatcggtccggcgctgaacatcgttaaacagggttacgaaggtaacttcatcggtgcgctggaaaccaccggtgttgttctgctgctggaatacatcccggaaatcaccctgccggttatcgcggcgctgtctatcgcggaatcttctacccagaaagaaaaaatcatcaaaaccatcgacaacttcctggaaaaacgttacgaaaaatggatcgaagtttacaaactggttaaagcgaaatggctgggtaccgttaacacccagttccagaaacgttcttaccagatgtaccgttctctggaataccaggttgacgcgatcaaaaaaatcatcgactacgaatacaaaatctactctggtccggacaaagaacagatcgcggacgaaatcaacaacctgaaaaacaaactggaagaaaaagcgaacaaagcgatgatcaacatcaacatcttcatgcgtgaatcttctcgttctttcctggttaaccagatgatcaacgaagcgaaaaaacagctgctggaattcgacacccagtctaaaaacatcctgatgcagtacatcaaagcgaactctaaattcatcggtatcaccgaactgaaaaaactggaatctaaaatcaacaaagttttctctaccccgatcccgttctcttactctaaaaacctggactgctgggttgacaacgaagaagacatcgacgttatcctgaaaaaatctaccatcctgaacctggacatcaacaacgacatcatctctgacatctctggtttcaactcttctgttatcacctacccggacgcgcagctggttccgggtatcaacggtaaagcgatccacctggttaacaacgaatcttctgaagttatcgttcacaaagcgatggacatcgaatacaacgacatgttcaacaacttcaccgtttctttctggctgcgtgttccgaaagtttctgcgtgccacctggaacagtacggtaccaacgaatactctatcatctcttctatgaaaaaacactctctgtctatcggttctggttggtctgtttctctgaaaggtaacaacctgatctggaccctgaaagactctgcgggtgaagttcgtcagatcaccttccgtgacctgccggacaaattcaacgcgtacctggcgaacaaatgggttttcatcaccatcaccaacgaccgtctgtgctctgcgaacctgtacatcaacggtgttctgatgggttctgcggaaatcaccggtctgggtgcgatccgtgaagacaacaacatcaccctgaaactggaccgttgcaacaacaacaaccagtacgtttctatcgacaaattccgtatcttctgcaaagcgctgaacccgaaagaaatcgaaaaactgtacacctcttacctgtctatcaccttcctgcgtgacttctggggtaacccgctgcgttacgacaccgaatactacctgatcccggttgcgtcttcttctaaagacgttcagctgaaaaacatcaccgactacatgtacctgaccaacgcgccgtgctacaccaacggtaaactgaacatctactaccgtcgtctgtacaacggtctgaaattcatcatcaaacgttacaccccgaacaacgaaatcgactgcttcgttaaatctggtgacttcatcaaactgtacgtttcttacaacaacaacgaacacatcgttggttacccgaaagacggtaacgcgttcaacaacctggaccgtatcctgcgtgttggttacaacgcgccgggtatcccgctgtacaaaaaaatggaagcggttaaactgcgtgacctgaaaacctactctgttcagctgaaactgtacgacgacaaaaacgcgtctctgggtctggttggtacccacaacggtcagatcggtaacgacccgaaccgtgacatcctgatcgcgtctaactggtacttcaaccacctgaaagacaaaatcctgggttgcgactggtacttcgttccgaccgacgaaggttggaccaacgactaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 7 - protein, 1314 aa, Clostridium tetani
PITINNFRYSDPVNNDTIIMMEPPYCKGLDIYYKAFKITDRIWIVPERYEFGTKPEDFNPPSSLIEGASEYYDPNYLRTDCDKDRFLQTMVKLFNRIKNNVAGEALLDKIINAIPYLGNCYSLLDKFDTNSNSVSFNLLEQDPCGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSCHEIIPSKQEIYMQHTYPISAEELFTFGGQDANLISIDIKNDLYEKTLNDYKAIANKLSQVTSCNDPNIDIDSYKQIYQQKYQFDKDCNGQYIVNEDKFQILYNSIMYGFTEIELGKKFNIKTRLSYFSMNHDPVKIPNLLDDTIYNDTEGFNIESKDLKSEYKGQNMRVNTNAFRNVDGCGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSACHLEQYGTNEYSIISSMKKHSLSIGSGWSVSLKGNNLIWTLKDSAGEVRQITFRDLPDKFNAYLANKWVFITITNDRLCSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPCYTNGKLNIYYRRLYNGLKFIIKRYTPNNEIDCFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 8 - DNA, 6816 bp, Artificial sequence
  vector pRSET-TeNT encoding the surface serine to cysteine substituted mature TeNT of SEQ ID NO 7
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccgagctcgagccgatcaccatcaacaacttccgttactctgacccggttaacaacgacaccatcatcatgatggaaccgccgtactgcaaaggtctggacatctactacaaagcgttcaaaatcaccgaccgtatctggatcgttccggaacgttacgaattcggtaccaaaccggaagacttcaacccgccgtcttctctgatcgaaggtgcgtctgaatactacgacccgaactacctgcgtaccgactgcgacaaagaccgtttcctgcagaccatggttaaactgttcaaccgtatcaaaaacaacgttgcgggtgaagcgctgctggacaaaatcatcaacgcgatcccgtacctgggtaactgctactctctgctggacaaattcgacaccaactctaactctgtttctttcaacctgctggaacaggacccgtgcggtgcgaccaccaaatctgcgatgctgaccaacctgatcatcttcggtccgggtccggttctgaacaaaaacgaagttcgtggtatcgttctgcgtgttgacaacaaaaactacttcccgtgccgtgacggtttcggttctatcatgcagatggcgttctgcccggaatacgttccgaccttcgacaacgttatcgaaaacatcacctctctgaccatcggtaaatctaaatacttccaggacccggcgctctgctgatgcacgaactgatccacgttctgcacggtctgtacggtatgcaggtttcttgccacgaaatcatcccgtctaaacaggaaatctacatgcagcacacctacccgatctctgcggaagaactgttcaccttcggtggtcaggacgcgaacctgatctctatcgacatcaaaaacgacctgtacgaaaaaaccctgaacgactacaaagcgatcgcgaacaaactgtctcaggttacctcttgcaacgacccgaacatcgacatcgactcttacaaacagatctaccagcagaaataccagttcgacaaagactgcaacggtcagtacatcgttaacgaagacaaattccagatcctgtacaactctatcatgtacggtttcaccgaaatcgaactgggtaaaaaattcaacatcaaaacccgtctgtcttacttctctatgaaccacgacccggttaaaatcccgaacctgctggacgacaccatctacaacgacaccgaaggtttcaacatcgaatctaaagacctgaaatctgaatacaaaggtcagaacatgcgtgttaacaccaacgcgttccgtaacgttgacggttgcggtctggtttctaaactgatcggtctgtgcaaaaaaatcatcccgccgaccaacatccgtgaaaacctgtacaaccgtaccgcgtctctgaccgacctgggtggtgaactgtgcatcaaaatcaaaaacgaagacctgaccttcatcgcggaaaaaaactctttctctgaagaaccgttccaggacgaaatcgtttcttacaacaccaaaaacaaaccgctgaacttcaactactctctggacaaaatcatcgttgactacaacctgcagtctaaaatcaccctgccgaacgaccgtaccaccccggttaccaaaggtatcccgtacgcgccggaatacaaatctaacgcggcgtctaccatcgaaatccacaacatcgacgacaacaccatctaccagtacctgtacgcgcagaaatctccgaccaccctgcagcgtatcaccatgaccaactctgttgacgacgcgctgatcaactctaccaaaatctactcttacttcccgtctgttatctctaaagttaaccagggtgcgcagggtatcctgttcctgcagtgggttcgtgacatcatcgacgacttcaccaacgaatcttctcagaaaaccaccatcgacaaaatctctgacgtttctaccatcgttccgtacatcggtccggcgctgaacatcgttaaacagggttacgaaggtaacttcatcggtgcgctggaaaccaccggtgttgttctgctgctggaatacatcccggaaatcaccctgccggttatcgcggcgctgtctatcgcggaatcttctacccagaaagaaaaaatcatcaaaaccatcgacaacttcctggaaaaacgttacgaaaaatggatcgaagtttacaaactggttaaagcgaaatggctgggtaccgttaacacccagttccagaaacgttcttaccagatgtaccgttctctggaataccaggttgacgcgatcaaaaaaatcatcgactacgaatacaaaatctactctggtccggacaaagaacagatcgcggacgaaatcaacaacctgaaaaacaaactggaagaaaaagcgaacaaagcgatgatcaacatcaacatcttcatgcgtgaatcttctcgttctttcctggttaaccagatgatcaacgaagcgaaaaaacagctgctggaattcgacacccagtctaaaaacatcctgatgcagtacatcaaagcgaactctaaattcatcggtatcaccgaactgaaaaaactggaatctaaaatcaacaaagttttctctaccccgatcccgttctcttactctaaaaacctggactgctgggttgacaacgaagaagacatcgacgttatcctgaaaaaatctaccatcctgaacctggacatcaacaacgacatcatctctgacatctctggtttcaactcttctgttatcacctacccggacgcgcagctggttccgggtatcaacggtaaagcgatccacctggttaacaacgaatcttctgaagttatcgttcacaaagcgatggacatcgaatacaacgacatgttcaacaacttcaccgtttctttctggctgcgtgttccgaaagtttctgcgtgccacctggaacagtacggtaccaacgaatactctatcatctcttctatgaaaaaacactctctgtctatcggttctggttggtctgtttctctgaaaggtaacaacctgatctggaccctgaaagactctgcgggtgaagttcgtcagatcaccttccgtgacctgccggacaaattcaacgcgtacctggcgaacaaatgggttttcatcaccatcaccaacgaccgtctgtgctctgcgaacctgtacatcaacggtgttctgatgggttctgcggaaatcaccggtctgggtgcgatccgtgaagacaacaacatcaccctgaaactggaccgttgcaacaaccaacaaccagtacgtttctatcgacaaattccgtatcttctgcaaagcgctgaacccgaaagaaatcgaaaaactgtacacctcttacctgtctatcaccttcctgcgtgacttctggggtaacccgctgcgttacgacaccgaatactacctgatcccggttgcgtcttcttctaaagacgttcagctgaaaaacatcaccgactacatgtacctgaccaacgcgccgtgctacaccaacggtaaactgaacatctactaccgtcgtctgtacaacggtctgaaattcatcatcaaacgttacaccccgaacaacgaaatcgactgcttcgttaaatctggtgacttcatcaaactgtacgtttcttacaacaacaacgaacacatcgttggttacccgaaagacggtaacgcgttcaacaacctggaccgtatcctgcgtgttggttacaacgcgccgggtatcccgctgtacaaaaaaatggaagcggttaaactgcgtgacctgaaaacctactctgttcagctgaaactgtacgacgacaaaaacgcgtctctgggtctggttggtacccacaacggtcagatcggtaacgacccgaaccgtgacatcctgatcgcgtctaactggtacttcaaccacctgaaagacaaaatcctgggttgcgactggtacttcgttccgaccgacgaaggttggaccaacgactaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 9 - protein, 1314 aa, Clostridium tetani
PITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVICKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSACHLEQYGTNEYSIISSMKKHSLSIGSGWSVSLKGNNLIWTLKDSAGEVRQITFRDLPDKFNAYLANKWVFITITNDRLCSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPCYTNGKLNIYYRRLYNGLKFIIKRYTPNNEIDCFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 10 - DNA, 6816 bp, Artificial sequence
  vector pRSET-TeNT encoding the surface serine to cysteine substituted mature TeNT of SEQ ID NO 9
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccgagctcgagccgatcaccatcaacaacttccgttactctgacccggttaacaacgacaccatcatcatgatggaaccgccgtactgcaaaggtctggacatctactacaaagcgttcaaaatcaccgaccgtatctggatcgttccggaacgttacgaattcggtaccaaaccggaagacttcaacccgccgtcttctctgatcgaaggtgcgtctgaatactacgacccgaactacctgcgtaccgactctgacaaagaccgtttcctgcagaccatggttaaactgttcaaccgtatcaaaaacaacgttgcgggtgaagcgctgctggacaaaatcatcaacgcgatcccgtacctgggtaactcttactctctgctggacaaattcgacaccaactctaactctgtttctttcaacctgctggaacaggacccgtctggtgcgaccaccaaatctgcgatgctgaccaacctgatcatcttcggtccgggtccggttctgaacaaaaacgaagttcgtggtatcgttctgcgtgttgacaacaaaaactacttcccgtgccgtgacggtttcggttctatcatgcagatggcgttctgcccggaatacgttccgaccttcgacaacgttatcgaaaacatcacctctctgaccatcggtaaatctaaatacttccaggacccggcgctgctgctgatgcacgaactgatccacgttctgcacggtctgtacggtatgcaggtttcttctcacgaaatcatcccgtctaaacaggaaatctacatgcagcacacctacccgatctctgcggaagaactgttcaccttcggtggtcaggacgcgaacctgatctctatcgacatcaaaaacgacctgtacgaaaaaaccctgaacgactacaaagcgatcgcgaacaaactgtctcaggttacctcttgcaacgacccgaacatcgacatcgactcttacaaacagatctaccagcagaaataccagttcgacaaagactctaacggtcagtacatcgttaacgaagacaaattccagatcctgtacaactctatcatgtacggtttcaccgaaatcgaactgggtaaaaaattcaacatcaaaacccgtctgtcttacttctctatgaaccacgacccggttaaaatcccgaacctgctggacgacaccatctacaacgacaccgaaggtttcaacatcgaatctaaagacctgaaatctgaatacaaaggtcagaacatgcgtgttaacaccaacgcgttccgtaacgttgacggttctggtctggtttctaaactgatcggtctgtgcaaaaaaatcatcccgccgaccaacatccgtgaaaacctgtacaaccgtaccgcgtctctgaccgacctgggtggtgaactgtgcatcaaaatcaaaaacgaagacctgaccttcatcgcggaaaaaaactctttctctgaagaaccgttccaggacgaaatcgtttcttacaacaccaaaaacaaaccgctgaacttcaactactctctggacaaaatcatcgttgactacaacctgcagtctaaaatcaccctgccgaacgaccgtaccaccccggttaccaaaggtatcccgtacgcgccggaatacaaatctaacgcggcgtctaccatcgaaatccacaacatcgacgacaacaccatctaccagtacctgtacgcgcagaaatctccgaccaccctgcagcgtatcaccatgaccaactctgttgacgacgcgctgatcaactctaccaaaatctactcttacttcccgtctgttatctgcaaagttaaccagggtgcgcagggtatcctgttcctgcagtgggttcgtgacatcatcgacgacttcaccaacgaatcttctcagaaaaccaccatcgacaaaatctctgacgtttctaccatcgttccgtacatcggtccggcgctgaacatcgttaaacagggttacgaaggtaacttcatcggtgcgctggaaaccaccggtgttgttctgctgctggaatacatcccggaaatcaccctgccggttatcgcggcgctgtctatcgcggaatcttctacccagaaagaaaaaatcatcaaaaccatcgacaacttcctggaaaaacgttacgaaaaatggatcgaagtttacaaactggttaaagcgaaatggctgggtaccgttaacacccagttccagaaacgttcttaccagatgtaccgttctctggaataccaggttgacgcgatcaaaaaaatcatcgactacgaatacaaaatctactctggtccggacaaagaacagatcgcggacgaaatcaacaacctgaaaaacaaactggaagaaaaagcgaacaaagcgatgatcaacatcaacatcttcatgcgtgaatcttctcgttctttcctggttaaccagatgatcaacgaagcgaaaaaacagctgctggaattcgacacccagtctaaaaacatcctgatgcagtacatcaaagcgaactctaaattcatcggtatcaccgaactgaaaaaactggaatctaaaatcaacaaagttttctctaccccgatcccgttctcttactctaaaaacctggactgctgggttgacaacgaagaagacatcgacgttatcctgaaaaaatctaccatcctgaacctggacatcaacaacgacatcatctctgacatctctggtttcaactcttctgttatcacctacccggacgcgcagctggttccgggtatcaacggtaaagcgatccacctggttaacaacgaatcttctgaagttatcgttcacaaagcgatggacatcgaatacaacgacatgttcaacaacttcaccgtttctttctggctgcgtgttccgaaagtttctgcgtgccacctggaacagtacggtaccaacgaatactctatcatctcttctatgaaaaaacactctctgtctatcggttctggttggtctgtttctctgaaaggtaacaacctgatctggaccctgaaagactctgcgggtgaagttcgtcagatcaccttccgtgacctgccggacaaattcaacgcgtacctggcgaacaaatgggttttcatcaccatcaccaacgaccgtctgtgctctgcgaacctgtacatcaacggtgttctgatgggttctgcggaaatcaccggtctgggtgcgatccgtgaagacaacaacatcaccctgaaactggaccgttgcaacaacaacaaccagtacgtttctatcgacaaattccgtatcttctgcaaagcgctgaacccgaaagaaatcgaaaaactgtacacctcttacctgtctatcaccttcctgcgtgacttctggggtaacccgctgcgttacgacaccgaatactacctgatcccggttgcgtcttcttctaaagacgttcagctgaaaaacatcaccgactacatgtacctgaccaacgcgccgtgctacaccaacggtaaactgaacatctactaccgtcgtctgtacaacggtctgaaattcatcatcaaacgttacaccccgaacaacgaaatcgactgcttcgttaaatctggtgacttcatcaaactgtacgtttcttacaacaacaacgaacacatcgttggttacccgaaagacggtaacgcgttcaacaacctggaccgtatcctgcgtgttggttacaacgcgccgggtatcccgctgtacaaaaaaatggaagcggttaaactgcgtgacctgaaaacctactctgttcagctgaaactgtacgacgacaaaaacgcgtctctgggtctggttggtacccacaacggtcagatcggtaacgacccgaaccgtgacatcctgatcgcgtctaactggtacttcaaccacctgaaagacaaaatcctgggttgcgactggtacttcgttccgaccgacgaaggttggaccaacgactaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 11 - protein, 1314 aa, Clostridium tetani; identical to SEQ ID NO: 7

SEQ ID NO: 12 - DNA, 6816 bp, Artificial sequence
  vector pRSET-TeNT encoding the surface serine to cysteine substituted mature TeNT of SEQ ID NO 11
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccgagctcgagccgatcaccatcaacaacttccgttactctgacccggttaacaacgacaccatcatcatgatggaaccgccgtactgcaaaggtctggacatctactacaaagcgttcaaaatcaccgaccgtatctggatcgttccggaacgttacgaattcggtaccaaaccggaagacttcaacccgccgtcttctctgatcgaaggtgcgtctgaatactacgacccgaactacctgcgtaccgactgcgacaaagaccgtttcctgcagaccatggttaaactgttcaaccgtatcaaaaacaacgttgcgggtgaagcgctgctggacaaaatcatcaacgcgatcccgtacctgggtaactgctactctctgctggacaaattcgacaccaactctaactctgtttctttcaacctgctggaacaggacccgtgcggtgcgaccaccaaatctgcgatgctgaccaacctgatcatcttcggtccgggtccggttctgaacaaaaacgaagttcgtggtatcgttctgcgtgttgacaacaaaaactacttcccgtgccgtgacggtttcggttctatcatgcagatggcgttctgcccggaatacgttccgaccttcgacaacgttatcgaaaacatcacctctctgaccatcggtaaatctaaatacttccaggacccggcgctgctgctgatgcacgaactgatccacgttctgcacggtctgtacggtatgcaggtttcttgccacgaaatcatcccgtctaaacaggaaatctacatgcagcacacctacccgatctctgcggaagaactgttcaccttcggtggtcaggacgcgaacctgatctctatcgacatcaaaaacgacctgtacgaaaaaaccctgaacgactacaaagcgatcgcgaacaaactgtctcaggttacctcttgcaacgacccgaacatcgacatcgactcttacaaacagatctaccagcagaaataccagttcgacaaagactgcaacggtcagtacatcgttaacgaagacaaattccagatcctgtacaactctatcatgtacggtttcaccgaaatcgaactgggtaaaaaattcaacatcaaaacccgtctgtcttacttctctatgaaccacgacccggttaaaatcccgaacctgctggacgacaccatctacaacgacaccgaaggtttcaacatcgaatctaaagacctgaaatctgaatacaaaggtcagaacatgcgtgttaacaccaacgcgttccgtaacgttgacggttgcggtctggtttctaaactgatcggtctgtgcaaaaaaatcatcccgccgaccaacatccgtgaaaacctgtacaaccgtaccgcgtctctgaccgacctgggtggtgaactgtgcatcaaaatcaaaaacgaagacctgaccttcatcgcggaaaaaaactctttctctgaagaaccgttccaggacgaaatcgtttcttacaacaccaaaaacaaaccgctgaacttcaactactctctggacaaaatcatcgttgactacaacctgcagtctaaaatcaccctgccgaacgaccgtaccaccccggttaccaaaggtatcccgtacgcgccggaatacaaatctaacgcggcgtctaccatcgaaatccacaacatcgacgacaacaccatctaccagtacctgtacgcgcagaaatctccgaccaccctgcagcgtatcaccatgaccaactctgttgacgacgcgctgatcaactctaccaaaatctactcttacttcccgtctgttatctctaaagttaaccagggtgcgcagggtatcctgttcctgcagtgggttcgtgacatcatcgacgacttcaccaacgaatcttctcagaaaaccaccatcgacaaaatctctgacgtttctaccatcgttccgtacatcggtccggcgctgaacatcgttaaacagggttacgaaggtaacttcatcggtgcgctggaaaccaccggtgttgttctgctgctggaatacatcccggaaatcaccctgccggttatcgcggcgctgtctatcgcggaatcttctacccagaaagaaaaaatcatcaaaaccatcgacaacttcctggaaaaacgttacgaaaaatggatcgaagtttacaaactggttaaagcgaaatggctgggtaccgttaacacccagttccagaaacgttcttaccagatgtaccgttctctggaataccaggttgacgcgatcaaaaaaatcatcgactacgaatacaaaatctactctggtccggacaaagaacagatcgcggacgaaatcaacaacctgaaaaacaaactggaagaaaaagcgaacaaagcgatgatcaacatcaacatcttcatgcgtgaatcttctcgttctttcctggttaaccagatgatcaacgaagcgaaaaaacagctgctggaattcgacacccagtctaaaaacatcctgatgcagtacatcaaagcgaactctaaattcatcggtatcaccgaactgaaaaaactggaatctaaaatcaacaaagttttctctaccccgatcccgttctcttactctaaaaacctggactgctgggttgacaacgaagaagacatcgacgttatcctgaaaaaatctaccatcctgaacctggacatcaacaacgacatcatctctgacatctctggtttcaactcttctgttatcacctacccggacgcgcagctggttccgggtatcaacggtaaagcgatccacctggttaacaacgaatcttctgaagttatcgttcacaaagcgatggacatcgaatacaacgacatgttcaacaacttcaccgtttctttctggctgcgtgttccgaaagtttctgcgtgccacctggaacagtacggtaccaacgaatactctatcatctcttctatgaaaaaacactctctgtctatcggttctggttggtctgtttctctgaaaggtaacaacctgatctggaccctgaaagactctgcgggtgaagttcgtcagatcaccttccgtgacctgccggacaaattcaacgcgtacctggcgaacaaatgggttttcatcaccatcaccaacgaccgtctgtgctctgcgaacctgtacatcaacggtgttctgatgggttctgcggaaatcaccggtctgggtgcgatccgtgaagacaacaacatcaccctgaaactggaccgttgcaacaacaacaaccagtacgtttctatcgacaaattccgtatcttctgcaaagcgctgaacccgaaagaaatcgaaaaactgtacacctcttacctgtctatcaccttcctgcgtgacttctggggtaacccgctgcgttacgacaccgaatactacctgatcccggttgcgtcttcttctaaagacgttcagctgaaaaacatcaccgactacatgtacctgaccaacgcgccgtgctacaccaacggtaaactgaacatctactaccgtcgtctgtacaacggtctgaaattcatcatcaaacgttacaccccgaacaacgaaatcgactgcttcgttaaatctggtgacttcatcaaactgtacgtttcttacaacaacaacgaacacatcgttggttacccgaaagacggtaacgcgttcaacaacctggaccgtatcctgcgtgttggttacaacgcgccgggtatcccgctgtacaaaaaaatggaagcggttaaactgcgtgacctgaaaacctactctgttcagctgaaactgtacgacgacaaaaacgcgtctctgggtctggttggtacccacaacggtcagatcggtaacgacccgaaccgtgacatcctgatcgcgtctaactggtacttcaaccacctgaaagacaaaatcctgggttgcgactggtacttcgttccgaccgacgaaggttggaccaacgactaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 13 - protein, 1314 aa, Clostridium tetani; identical to SEQ ID NO: 9

SEQ ID NO: 14 - DNA, 6816 bp, Artificial sequence; identical to SEQ ID NO: 10
  vector pRSET-TeNT encoding the surface serine to cysteine substituted mature TeNT of SEQ ID NO 13

(19) United States
(12) Patent Application Publication
(10) Pub. No.: US 2023/0057367 A1
(43) Pub. Date: Feb. 23, 2023

(54) COMPOSITION AND METHOD

(71) Applicant: SNORETOX LTD, Kew, Victoria (AU)

(72) Inventors: Thomas McLean, Melbourne (AU); Peter Smooker, Melbourne (AU); Luke Norbury, Melbourne (AU); Peter Coloe, Melbourne (AU); Russell Conduit, Melbourne (AU); Anthony Sasse, Kew (AU)

(21) Appl. No.: 17/796,976

(22) PCT Filed: Feb. 3, 2021
(86) PCT No.: PCT/AU2021/050078

§ 371 Date: Aug. 2, 2022

(30) Foreign Application Priority Data
Feb. 3, 2020 (AU) 2020900283

(51) Int. Cl. A61K 47/64; A61P 21/00

(52) U.S. Cl. CPC A61K 47/64; A61P 21/00

(57) ABSTRACT

The invention relates to a tetanus neurotoxin (TeNT) conjugate comprising a TeNT conjugated to at least one masking moiety (i) through an acid labile linkage or (ii) comprising a masking polypeptide. The invention also relates to a composition comprising the TeNT conjugate and therapeutic use of the TeNT conjugate or composition for treating hypotonia in a subject or enhancing muscle power and/or muscle tone and/or muscle healing and/or sporting performance.

### FIELD

The present disclosure relates to a tetanus neurotoxin (TeNT) conjugate or a composition comprising the TeNT conjugate. The disclosure also relates to therapeutic use of the TeNT conjugate or composition.

### BACKGROUND

Tetanus neurotoxin (TeNT) is produced by Clostridium tetani. TeNT acts at the spinal cord and blocks release at the spinal inhibitory interneurons of γ-aminobutyric acid (GABA) and glycine, which are inhibitory neurotransmitters. As such, TeNT causes spastic paralysis. TeNT does not occur in multiple serotypes.

Exploitation of biological properties of TeNT, or at least fragments of TeNT, for therapy has been proposed. However, long-term treatment with protein therapeutic agents tends to lead to a targeted immune response. As there is only one serotype of TeNT known, serotype switching to circumvent immunity is not an option for TeNT-based therapy. Moreover, many populations are vaccinated against TeNT, thereby precluding TeNT-based therapy.

US 2002/0197278 A1 discloses use of PEGylated botulinum toxins for treating disorders of inappropriate muscle contraction. US 2002/0197278 A1 also suggests use of PEGylated TeNT for treating disorders of inappropriate muscle contraction, e.g. migration headache or strabismus. However, as noted above, TeNT causes muscle contraction, thereby precluding its use, PEGylated or not, for treating disorders of inappropriate muscle contraction.

Wan et al. Process Biochemistry (2017) 52: 183-191 discloses the effect of PEGylation on the anti-PEG immune response resulting from administration of PEGylated proteins, but does not exploit its findings for any therapy.

WO 2016/001762 A1 discloses use of a PEGylated TeNT fragment c (c) for increasing muscle mass. Fragment c (50 kDa) is generated when TeNT is enzymatically cleaved by papain and corresponds to the 451 amino acids at the C-terminus of the TeNT heavy chain. Fragment c retains the binding, internalization and trans-synaptic transport capabilities of undigested TeNT, but does not disrupt any neuronal processes, and is therefore nontoxic.

A need exists for a TeNT-based therapy that avoids the pre-existing anti-TeNT immune response in tetanus toxoid immunised subjects.

It is to be understood that if any prior art publication is referred to herein, such reference does not constitute an admission that the publication forms a part of the common general knowledge in the art in Australia or any other country.

### SUMMARY

The inventors have appreciated that exploitation of TeNT has not been fully realised because of the adaptive immune system, which upon administration of a protein therapeutic agent, produces an antibody response, thereby decreasing efficacy of the protein therapeutic agent. The adaptive immune response may be intentional, as a result of vaccination, as demonstrated by many populations that have been immunised against TeNT. Alternatively, the adaptive immune response may be unintentional, resulting from repeated exposure to the protein therapeutic agent.

The inventors have produced a family of modified TeNTs and a treatment regimen that address these problems.

Specifically, the invention provides a family of PEGylated TeNTs (PEG-TeNTs) or Peptide-masked TeNTs (Pep-TeNTs) that each evade the immune system until the TeNT has reached the active site in the central nervous system. The incorporation of acid labile linkages between the TeNT and the masking moieties (e.g., PEG or repeating peptides) confers protection from the immune response until a reduction in pH in transport vesicle, upon entering the inhibitory interneuron cytosol, causes a dissociation of the masking agents from the TeNT. This system allows for the delivery of a highly-active TeNT to the central nervous system (CNS) that is immune-evading prior to uptake, making it effective for the treatment of muscle hypotonia in individuals with a protective antibody response.

A first aspect provides a tetanus neurotoxin conjugate, comprising a tetanus neurotoxin (TeNT) conjugated to at least one masking moiety through an acid labile linkage.

In one embodiment of the first aspect, the masking moiety is a polyethylene glycol (PEG).

In one embodiment of the first aspect, the masking moiety is a masking polypetide.

A second aspect provides a tetanus neurotoxin conjugate comprising TeNT conjugated to at least one masking moiety, the masking moiety comprising a masking polypetide.

In one embodiment, the masking polypeptide comprises a short repeating peptide sequence. In one embodiment, the short repeating peptide sequence comprises glycine and threonine.

In one embodiment of the second aspect, the tetanus neurotoxin conjugate is conjugated to at least one masking moiety through an acid labile linkage.

In one embodiment, the masking moiety is linked to the TeNT light chain (LC), or the TeNT heavy chain (HC), or the TeNT fragment c (c).

In one embodiment, the TeNT light chain (LC) is PEGylated, the TeNT heavy chain (HC) is PEGylated, or the TeNT fragment c (c) is PEGylated. In one embodiment, LC is PEGylated and HC is PEGylated (PEG-TeNT-LC-HC), or LC is PEGylated and c is PEGylated (PEG-TeNT-LC-c).

In one embodiment, the PEG is conjugated to a lysine residue of TeNT. In another embodiment, the PEG is conjugated to a cysteine residue of TeNT. In one embodiment, the PEG is conjugated to a cysteine residue of TeNT, wherein the cysteine residue is either native or introduced, optionally by substitution for a serine residue relative to SEQ ID NO: 1.

In one embodiment, the PEG has a molecular weight of about 5 kDa, about 10 kDa, or about 20 kDa, or about 30 kDa.

In one embodiment, the masking polypetide is conjugated to a lysine residue of TeNT. In another embodiment, the masking polypetide is conjugated to a cysteine residue of TeNT. In one embodiment, the masking polypetide is conjugated to a cysteine residue of TeNT, wherein the cysteine residue is either native or introduced, optionally by substitution for a serine residue relative to SEQ ID NO: 1.

In one embodiment, the masking polypetide comprises a short, repeating amino acid sequence and is conjugated to a lysine residue of TeNT. In another embodiment, the masking polypetide comprises a short, repeating amino acid sequence and is conjugated to a cysteine residue of TeNT. In one embodiment, the masking polypetide comprises a short, repeating amino acid sequence and is conjugated to a cysteine residue of TeNT, wherein the cysteine residue is either native or introduced, optionally by substitution for a serine residue relative to SEQ ID NO: 1.

In one embodiment, the masking polypetide has a molecular weight of about 2 kDa, about 5 kDa, about 10 kDa, or about 20 kDa, or about 30 kDa.

A third aspect provides a composition comprising the tetanus neurotoxin conjugate of the first or second aspect.

A fourth aspect provides a composition comprising:
(a) a first tetanus conjugate comprising:
   (i) a tetanus neurotoxin (TeNT) or fragment thereof, conjugated to at least one masking moiety through an acid labile linkage; or
   (ii) a tetanus neurotoxin (TeNT) or fragment thereof, conjugated to at least one masking polypeptide through a non-acid labile linkage; and
(b) a tetanus neurotoxin or a second tetanus neurotoxin conjugate.

In one embodiment, the composition is a therapeutic composition.

In another embodiment, the composition is a cosmetic composition.

A fifth aspect provides a method for treating hypotonia, the method comprising administering to a subject a tetanus neurotoxin conjugate of the first or second aspect, or a composition of the third or fourth aspect.

A fifth aspect alternatively provides a tetanus neurotoxin conjugate of the first or second aspect, or a composition of the third or fourth aspect, for use in treating hypotonia; or use of a tetanus neurotoxin conjugate of the first or second aspect, or a composition of the third or fourth aspect, in the manufacture of a medicament for treating hypotonia.

A sixth aspect provides a method for treating hypotonia in a subject, the method comprising administering to a subject a tetanus neurotoxin conjugate of the first or second aspect, and a tetanus neurotoxin, or a composition of the fourth aspect.

A sixth aspect alternatively provides a tetanus neurotoxin conjugate of the first or second aspect, and a tetanus neurotoxin, or a composition of the fourth aspect, for use in treating hypotonia in a subject; or use of a tetanus neurotoxin conjugate of the first or second aspect, and a tetanus neurotoxin, or a composition of the fourth aspect, in the manufacture of a medicament for treating hypotonia in a subject.

In one embodiment, the hypotonia is obstructive sleep apnoea.

A seventh aspect provides a method for enhancing muscle tone, muscle power, muscle healing and/or sporting performance, the method comprising administering to a subject a tetanus neurotoxin conjugate of the first or second aspect, or a composition of the third or fourth aspect.

A seventh aspect alternatively provides a tetanus neurotoxin conjugate of the first or second aspect, or a composition of the third or fourth aspect, for use in enhancing muscle tone, muscle power, muscle healing and/or sporting performance; or use of a tetanus neurotoxin conjugate of the first or second aspect, or a composition of the third or fourth aspect, in the manufacture of a medicament for enhancing muscle tone, muscle power, muscle healing and/or sporting performance.

In one embodiment, the first TeNT conjugate or the second TeNT conjugate comprises a PEGylated TeNT light chain (LC), a PEGylated TeNT heavy chain (HC), a PEGylated TeNT heavy chain (HC) and PEGylated TeNT light chain (LC), or a PEGylated TeNT fragment c (c). In one embodiment, the first TeNT conjugate or the second TeNT conjugate comprises PEG-TeNT-LC-HC.

In another embodiment, the first TeNT conjugate or the second TeNT conjugate is PEG-TeNT-HC comprising a PEGylated HC. In this embodiment, LC is not PEGylated. In another embodiment, the first TeNT conjugate or the second TeNT conjugate is PEG-TeNT-LC-c comprising a PEGylated LC and a PEGylated c. In this embodiment, HN is not PEGylated.

In a further embodiment, the first TeNT conjugate is PEG-TeNT-HC, and the second TeNT conjugate is PEG-TeNT-LC-c.

In one embodiment, treating comprises administering to the subject: a TeNT conjugate comprising PEGylated c (PEG-TeNT-c) until efficacy decreases; then a composition comprising PEG-TeNT-HC and PEG-TeNT-LC-c until efficacy decreases; then a TeNT conjugate comprising a PEGylated LC and a PEGylated HC (PEG-TeNT-LC-HC).

In one embodiment, treating comprises administering to the subject: a TeNT conjugate comprising PEGylated c (PEG-TeNT-c); then a composition comprising PEG-TeNT-HC and PEG-TeNT-LC-c; then a TeNT conjugate comprising a PEGylated LC and a PEGylated HC (PEG-TeNT-LC-HC), to determine the immunological profile of anti-TeNT antibodies of the subject and determine the effective composition of TeNT conjugates based on that profile.

In one embodiment, treating comprises administering to the subject a first TeNT conjugate comprising a PEGylated HC (PEG-TeNT-HC) and a second TeNT conjugate comprising a PEGylated LC and a PEGylated c (PEG-TeNT-LC-c).

In another embodiment, treating comprises administering to the subject: a TeNT conjugate comprising a PEGylated c (PEG-TeNT-c); and/or a first TeNT conjugate comprising a PEGylated HC (PEG-TeNT-HC) and a second TeNT conjugate comprising a PEGylated LC-c (PEG-TeNT-LC-c); and/or a TeNT conjugate comprising a PEGylated HC and a PEGylated LC (PEG-TeNT-LC-HC).

In one embodiment, treating comprises administering to the subject: a TeNT conjugate comprising PEGylated c (PEG-TeNT-c) until efficacy decreases. Thereafter, treating may comprise administering to the subject: a PEG-TeNT-HC and a PEG-TeNT-LC-c until efficacy decreases. Thereafter, treating may comprise administering to the subject: a TeNT conjugate comprising a PEGylated LC and a PEGylated HC (PEG-TeNT-LC-HC).

In one embodiment, treating comprises administering to the subject: a TeNT conjugate comprising PEGylated c (PEG-TeNT-c) until efficacy decreases; then a PEG-TeNT-HC and a PEG-TeNT-LC-c until efficacy decreases; then a TeNT conjugate comprising a PEGylated LC and a PEGylated HC (PEG-TeNT-LC-HC).

In one embodiment, treating comprises administering to the subject: a TeNT conjugate comprising PEGylated c (PEG-TeNT-c) comprising PEG having a molecular weight of about 5 kDa until efficacy decreases; then a PEG-TeNT comprising PEGylated c (PEG-TeNT-c) comprising PEG having a molecular weight of about 10 kDa until efficacy decreases; then a TeNT conjugate comprising PEGylated c (PEG-TeNT-c) comprising PEG having a molecular weight of about 20 kDa until efficacy decreases. Thereafter, treating may comprise administering to the subject: a PEG-TeNT-HC and a PEG-TeNT-LC-c, either or both of which comprise PEG having a molecular weight of 5 kDa, until efficacy decreases; then a PEG-TeNT-HC and a PEG-TeNT-LC-c, either or both of which comprise PEG having a molecular weight of 10 kDa, until efficacy decreases; then a PEG-TeNT-HC and a PEG-TeNT-LC-c, either or both of which comprise PEG having a molecular weight of 20 kDa, until efficacy decreases. Thereafter, treating may comprise administering to the subject: a TeNT conjugate comprising a PEGylated LC and a PEGylated HC (PEG-TeNT-LC-HC), either or both of which comprise PEG having a molecular weight of 5 kDa, until efficacy decreases; then a TeNT conjugate comprising a PEGylated LC and a PEGylated HC (PEG-TeNT-LC-HC), either or both of which comprise PEG having a molecular weight of 10 kDa, until efficacy decreases; then a PEG-TeNT comprising a PEGylated LC and a PEGylated HC (PEG-TeNT-LC-HC), either or both of which comprise PEG having a molecular weight of 20 kDa.

In one embodiment, treating comprises administering to the subject: a TeNT conjugate comprising PEGylated c (PEG-TeNT-c); then a composition comprising PEG-TeNT-HC and PEG-TeNT-LC-c; then a TeNT conjugate comprising a PEGylated LC and a PEGylated HC (PEG-TeNT-LC-HC), to determine the immunological profile of anti-TeNT antibodies of the subject and determine the effective composition of TeNT conjugates based on that profile.

In one embodiment, treating comprises administering to the subject a first TeNT conjugate comprising a masking polypeptide conjugated to HC (PEP-TeNT-HC) and a second TeNT conjugate comprising a masking polypeptide conjugated to LC and a masking polypeptide conjugated to fragment c (PEP-TeNT-LC-c).

In another embodiment, treating comprises administering to the subject: a TeNT conjugate comprising a masking polypeptide conjugated to fragment c (PEP-TeNT-c); and/or a first TeNT conjugate comprising a masking polypeptide conjugated to HC (PEP-TeNT-HC) and a second TeNT conjugate comprising a masking polypeptide conjugated to LC-c (PEP-TeNT-LC-c); and/or a TeNT conjugate comprising a masking polypeptide conjugated to HC and a masking polypeptide conjugated to LC (PEP-TeNT-LC-HC).

In one embodiment, treating comprises administering to the subject: a TeNT conjugate comprising a masking polypeptide conjugated to c (PEP-TeNT-c) until efficacy decreases. Thereafter, treating may comprise administering to the subject: a PEP-TeNT-HC and a PEP-TeNT-LC-c until efficacy decreases. Thereafter, treating may comprise administering to the subject: a TeNT conjugate comprising a masking polypeptide conjugated to LC and a masking polypeptide conjugated to HC (PEP-TeNT-LC-HC).

In one embodiment, treating comprises administering to the subject: a TeNT conjugate comprising a masking polypeptide conjugated to c (PEP-TeNT-c) until efficacy decreases; then a PEP-TeNT-HC and a PEP-TeNT-LC-c until efficacy decreases; then a TeNT conjugate comprising a masking polypeptide conjugated to LC and a masking polypeptide conjugated to HC (PEP-TeNT-LC-HC).

In one embodiment, treating comprises administering to the subject: a TeNT conjugate comprising fragment c conjugated to a masking polypeptide (PEP-TeNT-c), the masking polypeptide comprising a peptide of short, repeating amino acid sequences having a molecular weight of about 5 kDa until efficacy decreases; then a TeNT conjugate comprising fragment c conjugated to a masking polypeptide (PEP-TeNT-c), the masking polypeptide comprising a peptide of short, repeating amino acid sequences having a molecular weight of about 10 kDa until efficacy decreases; then a TeNT conjugate comprising fragment c conjugated to a masking polypeptide (PEP-TeNT-c), the masking polypeptide comprising a peptide of short, repeating amino acid sequences having a molecular weight of about 20 kDa until efficacy decreases. Thereafter, treating may comprise administering to the subject: a PEP-TeNT-HC and a PEP-TeNT-LC-c, either or both of which comprise a peptide of short, repeating amino acid sequences having a molecular weight of about 5 kDa, until efficacy decreases; then a PEP-TeNT-HC and a PEP-TeNT-LC-c, either or both of which comprise a PEP having a molecular weight of about 10 kDa, until efficacy decreases; then a PEP-TeNT-HC and a PEP-TeNT-LC-c, either or both of which comprising a peptide of short, repeating amino acid sequences having a molecular weight of about 20 kDa, until efficacy decreases. Thereafter, treating may comprise administering to the subject: a TeNT conjugate comprising a masking polypeptide conjugated to LC and a masking polypeptide conjugated to HC (PEP-TeNT-LC-HC), of which either or both masking polypeptides comprise a peptide of short, repeating amino acid sequences having a molecular weight of about 5 kDa, until efficacy decreases; then a TeNT conjugate comprising a masking polypeptide conjugated to LC and a masking polypeptide conjugated to HC (PEP-TeNT-LC-HC), either or both conjugates comprising a masking polypeptide having a molecular weight of about 10 kDa, until efficacy decreases; then a TeNT conjugate comprising a masking polypeptide conjugated to LC and a masking polypeptide conjugated to HC (PEP-TeNT-LC-HC), either or both of which comprise a peptide of short, repeating amino acid sequences having a molecular weight of about 20 kDa.

In one embodiment, treating comprises administering to the subject: a TeNT conjugate comprising a masking polypeptide conjugated to c (PEP-TeNT-c); then a composition comprising PEP-TeNT-HC and PEP-TeNT-LC-c; then a TeNT conjugate comprising a masking polypeptide conjugated to LC and a masking polypeptide conjugated to HC (PEP-TeNT-LC-HC), to determine the immunological profile of anti-TeNT antibodies of the subject and determine the effective composition of TeNT conjugates based on that profile.

A tenth aspect provides a kit comprising the TeNT of the first or second aspect, the composition of the third or fourth aspect.

### BRIEF DESCRIPTION OF THE FIGURES

FIG. 1 is a diagrammatic representation of hydrazone PEGylated, PEPylated and Hydrazone PEPylated TeNTs: (A) PEG-HZN-TeNT-c; (B) PEP-TeNT-HC; (C) PEP-TeNT-LC-c; (D) PEP-TeNT-LC-HC; (E) PEP-HZN-TeNT-LC-HC; (F) PEP-TeNT-c; (G) PEG-HZN-TeNT-HC; (H) PEG-HZN-TeNT-LC-c; (I) PEG-HZN-TeNT-LC-HC.

FIG. 2 is a representative chemical reaction for the addition of PEG to TeNT by a pH-labile hydrazone linkage.

FIG. 3 is a representative chemical reaction for the addition of masking Glycopeptide to TeNT by a pH-labile hydrazone linkage.

FIG. 4 is a representative chemical reaction for the addition of masking peptide to TeNT.

FIG. 5 is a schematic representation of example PEG-TeNTs of the disclosure: (A) PEG-TeNT-c; (B) PEG-TeNT-HC; (C) PEG-TeNT-LC-c; (D) PEG-TeNT-LC-HC.

FIG. 6 is the amino acid sequence (SEQ ID NO: 1) of mature TeNT comprising 1314 amino acids.

FIG. 7 is a nucleic acid sequence (SEQ ID NO: 2) of the vector pRSET-TeNT encoding TeNT.

FIG. 8 is a map of the vector pRSET-TeNT encoding TeNT. TeNT is expressed with an N-terminal His₆-tag. The nucleic acid was inserted within the multiple cloning site (MCS) of the pRSET-A vector and expressed under the control of a T7 promoter.

FIG. 9 is the amino acid sequence (SEQ ID NO: 3) of HC comprising amino acids 457 to 1314 of SEQ ID NO: 1.

FIG. 10 is the amino acid sequence (SEQ ID NO: 4) of c comprising amino acids 864 to 1314 of SEQ ID NO: 1.

FIG. 11 is the amino acid sequence (SEQ ID NO: 5) of a mature 1314 amino acid TeNT comprising surface serine to cysteine amino acid substitutions S81C, S120C, S144C, S248C, S335C, S428C, S600C, S963C, S1041C, S1155C, and S1187C relative to SEQ ID NO: 1.

FIG. 12 is a nucleic acid sequence (SEQ ID NO: 6) of the vector pRSET-TeNT encoding the surface serine to cysteine substituted mature TeNT of FIG. 11 (SEQ ID NO: 5).

FIG. 13 is a map of the vector pRSET-TeNT of FIG. 12 (SEQ ID NO: 6) encoding the surface serine to cysteine substituted mature TeNT of FIG. 11 (SEQ ID NO: 5). The surface serine to cysteine substituted TeNT is expressed with an N-terminal His₆-tag. The nucleic acid was inserted within the MCS of the pRSET-A vector and expressed under the control of a T7 promoter.

FIG. 14 is the amino acid sequence (SEQ ID NO: 7) of a mature 1314 amino acid TeNT comprising surface serine to cysteine amino acid substitutions S81C, S120C, S144C, S248C, S335C, S428C, S963C, S1041C, S1155C, and S1187C, relative to SEQ ID NO: 1, in the LC and c regions.

FIG. 15 is a nucleic acid sequence (SEQ ID NO: 8) of the vector pRSET-TeNT encoding the surface serine to cysteine substituted mature TeNT of FIG. 14 (SEQ ID NO: 7).

FIG. 16 is a map of the vector pRSET-TeNT of FIG. 15 (SEQ ID NO: 8) encoding the surface serine to cysteine substituted mature TeNT of 14 (SEQ ID NO: 7). The surface serine to cysteine substituted TeNT is expressed with an N-terminal His₆-tag. The nucleic acid was inserted within the MCS of the pRSET-A vector and expressed under the control of a T7 promoter.

FIG. 17 is the amino acid sequence (SEQ ID NO: 9) of a TeNT comprising HC surface serine to cysteine amino acid substitutions S600C, S963C, S1041C, S1155C, and S1187C relative to SEQ ID NO: 1.

FIG. 18 is a nucleic acid sequence (SEQ ID NO: 10) of the vector pRSET-TeNT encoding the surface serine to cysteine substituted mature TeNT of FIG. 17 (SEQ ID NO: 9).

FIG. 19 is a map of the vector pRSET-TeNT of 18 (SEQ ID NO: 10) encoding the surface serine to cysteine substituted TeNT of FIG. 17 (SEQ ID NO: 9). The surface serine to cysteine substituted TeNT is expressed with an N-terminal His₆-tag. The nucleic acid was inserted within the MCS of the pRSET-A vector and expressed under the control of a T7 promoter.

FIG. 20 is the amino acid sequence (SEQ ID NO: 11) of a mature 1314 amino acid TeNT comprising surface serine to cysteine amino acid substitutions S81C, S120C, S144C, S248C, S335C, S428C, S963C, S1041C, S1155C, and S1187C, relative to SEQ ID NO: 1, in the LC and c regions.

FIG. 21 is a nucleic acid sequence (SEQ ID NO: 12) of the vector pRSET-TeNT encoding the surface serine to cysteine substituted mature TeNT of FIG. 20 (SEQ ID NO: 11).

FIG. 22 is a map of the vector pRSET-TeNT of FIG. 21 (SEQ ID NO: 12) encoding the surface serine to cysteine substituted mature TeNT of FIG. 20 (SEQ ID NO: 11). The surface serine to cysteine substituted TeNT is expressed with an N-terminal His₆-tag. The nucleic acid was inserted within the MCS of the pRSET-A vector and expressed under the control of a T7 promoter.

FIG. 23 is the amino acid sequence (SEQ ID NO: 13) of a TeNT comprising HC surface serine to cysteine amino acid substitutions S600C, S963C, S1041C, S1155C, and S1187C relative to SEQ ID NO: 1.

FIG. 24 is a nucleic acid sequence (SEQ ID NO: 14) of the vector pRSET-TeNT encoding the surface serine to cysteine substituted mature TeNT of FIG. 23 (SEQ ID NO: 13).

FIG. 25 is a map of the vector pRSET-TeNT of FIG. 24 (SEQ ID NO: 14) encoding the surface serine to cysteine substituted TeNT of FIG. 23 (SEQ ID NO: 13). The surface serine to cysteine substituted TeNT is expressed with an N-terminal His₆-tag. The nucleic acid was inserted within the MCS of the pRSET-A vector and expressed under the control of a T7 promoter.

FIG. 26 is a 3-dimensional protein structure model of TeNT derived from crystallography data deposited in the protein data bank (accession ID PDB: 5N0B) mapping epitopes recognised by major human antibody clonotypes, as identified by da Silva Antunes et al. (2017) and Palermo et al. (2017), onto the model using Discovery Studio. Surface serine residues in or around the identified epitopes were selected for mutation to cysteine for subsequent PEGylation.

FIG. 27 is (A) a photograph of an SDS-PAGE gel demonstrating the attachment of 5 kDa PEG-Aldehyde to surface lysine residues of TeNT by a heter-bifunctional cross-linker, Succinimidyl 6-hydrazinonicotinamide acetone hydrazone (SANH), that introduces an acid-labile hydrazone linkage between the PEG and TeNT (PEG-HZN-TeNT-LC-HC) and (B) a photograph of an SDS-PAGE gel demonstrating the PEG-HZN-TeNT-LC-HC after trypsin digestion to the active form.

### DETAILED DESCRIPTION

The present invention relates in one form to immune-evading TeNT conjugates which comprise an immune masking moiety that is released in an acidic environment. The present invention also relates to compositions comprising the immune-evading TeNT conjugates, and their therapeutic and cosmetic use. The masking moieties function prior to uptake of the TeNT into neurons, and are released from the conjugate following uptake into the CNS inhibitory interneuron, resulting in release of active TeNT to the active site in the CNS. Without wishing to be bound by theory, the inventors understand that this happens in the vesicle in which the masked TeNT is transported from the peripheral nerve to the CNS. When the vesicle enters the inhibitory neuron, it acidifies. The TeNT then undergoes a conformation change that releases the active TeNT into the cytosol. In one embodiment, the inventors have exploited this using acid-sensitive masking.

In one embodiment, the invention relates to treating hypotonia, optionally obstructive sleep apnoea.

Described herein is use of a TeNT conjugate to treat hypotonia in subjects with a protective immune response against tetanus toxoid. To achieve that end, an active TeNT is conjugated to a masking moiety, such as PEG or a masking polypeptide through an acid labile linkage. Under acidic conditions within the inhibitory interneuronal cells, the acid labile linkage is hydrolysed or cleaved to release the active TeNT. The TeNT conjugates described herein may therefore be used for any function for which administering active tetanus neurotroxin is useful. For example, the conjugates may be used for increasing muscle tone in a subject, such as, for example, tetanus-immune patients.

The activity of the TeNT conjugates described herein can be demonstrated by the administration of a unit defined dose of conjugate, or composition thereof, where at the same unit dose TeNT would exhibit no activity or reduced activity in a vaccinated subject. In some embodiments, mutations are introduced into the TeNT for attachment of the masking moiety. The introduction of specific surface mutations for the directed attachment of masking moieties, such as polypeptides or PEG molecules, based on the analysis of the three-dimensional structure of TeNT, allows the masking of specific TeNT epitopes known to be targeted by the protective antibody response in vaccinated subjects. The combinations of pH-dependent PEGylation, polypeptide conjugation and site-directed mutation greatly increased the effect of the molecule on increasing muscle tone in vaccinated mammalian models, relative to the equivalent units administered of TeNT.

The inventors further envisage that increasing muscle tone will enhance muscle muscle recovery and healing, muscle power and tone, and ultimately enhanced sports performance. Accordingly, also described herein is use of TeNT conjugates to enhance muscle recovery, enhance muscle power and tone, and enhance sports performance.

US 2002/0197278 disclosed a series of PEGylated botulinum toxins for treating disorders of inappropriate muscle contraction and suggested that TeNT could be used as an alternative to botulium toxin. However, TeNT cannot be used to treat muscle contraction. Further, the alleged invention of US 2002/0197278 appears not to be enabled, because the methods disclosed do not include site directed masking of epitopes, and, to the best of the present inventors' knowledge, the three-dimensional structure and identification of epitopes required for deliberate masking of TeNT epitopes was not available at the priority date of US 2002/0197278.

Wan et al. is directed to the effect of PEGylation on the anti-PEG immune response resulting from administration of PEGylated proteins, but does not exploit its findings for any therapy. Although Wan et al. disclose that a PEGylated tetanus toxoid demonstrated reduced immunogenicity relative to non-PEGylated tetanus toxoid, Wan et al. do not present a therapeutically relevant molecule or formulation. Moreover, PEGylation of tetanus toxoid is not relevant to modification of active TeNT, because tetanus toxoid is a biologically inactive TeNT used for vaccination, which may be produced by formaldehyde cross-linking of TeNT. That is, tetanus toxoid, PEGylated or not, does not possess the combination of enzymatic, binding and translocational activity of active TeNT.

The person skilled in the art will understand that PEGylating TeNT is a different process to the masking of the present disclosure, because PEGylating does not require retention of activity of the target molecule, e.g. TeNT. In contrast, the present disclosure requires masking in a way that reduces immunogenicity while retaining TeNT activity. The person skilled in the art will understand that the latter is more difficult than the former.

WO 2016/001762 A1 relates to a TeNT c-fragment alone, which is a molecule with no specific activity beyond binding neurotransmitters and entering the neuron.

A need exists for improved TeNT-based therapy that avoids the pre-existing anti-TeNT immunity in tetanus toxoid immunised subjects. Disclosed herein is a masked, active and therapeutically relevant TeNT conjugate comprising TeNT conjugated to masking moieties, typically through an acid labile linkage.

Tetanus Neurotoxin (TeNT)

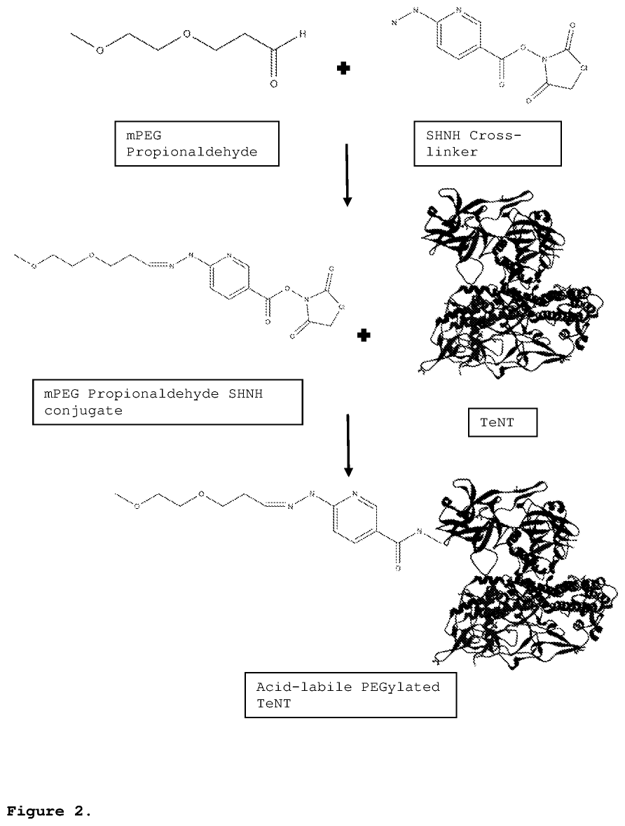

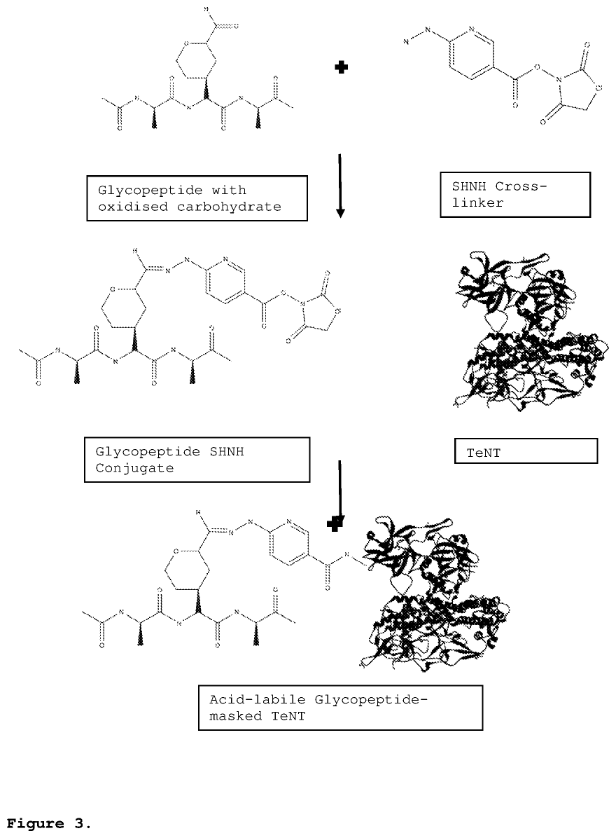

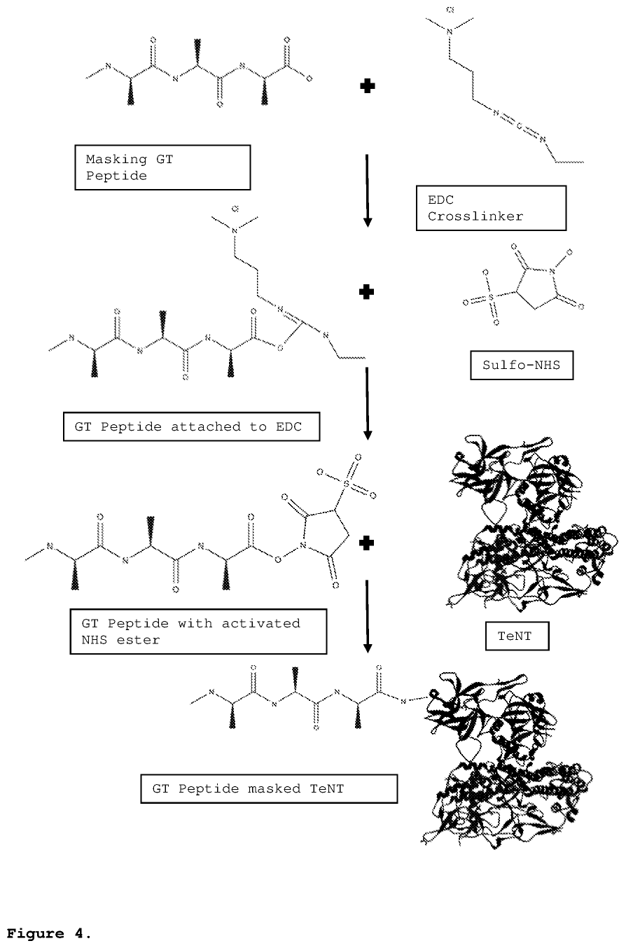

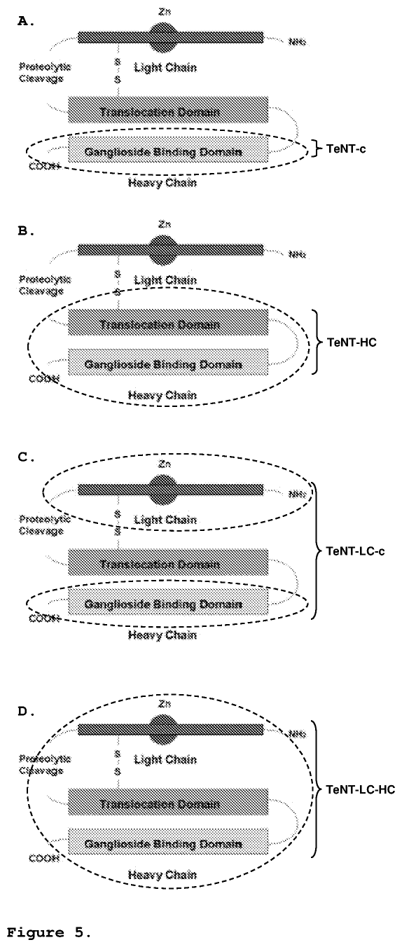

TeNT is approximately 150 kDa and is expressed from the tetX gene. A codon optimised nucleic acid sequence corresponding to the coding region of tetX, but lacking the initiator methionine codon, is provided in the vector sequence of FIG. 3 (SEQ ID NO: 2). TeNT is expressed as one protein that is post-translationally cleaved—first to remove the initiator methionine and then into two parts: a 50 kDa light chain (LC or A-chain) derived from the N-terminus of the uncleaved protein and a 100 kDa heavy chain (HC or B-chain) derived from the C-terminus of the uncleaved protein. The two chains are connected by an interchain disulfide bond, which is essential for neurotoxicity. The 1314 amino acid sequence of mature TeNT is provided in FIG. 2 (SEQ ID NO: 1).

LC has zinc endopeptidase activity and attacks the vesicle-associated membrane protein (VAMP) that is necessary for vesicle fusion to membranes, thereby preventing neurotransmitter release.

Upon digestion with papain, HC can be cleaved into two domains, each of 50 kDa: an N-terminus translocation domain named HN; and a C-terminus ganglioside (membrane) binding domain named fragment c (c). TeNT lacking c is referred to herein as LC-HN.

c harbours two polysialoganglioside binding sites and binds to polysialogangliosides (GD2 GD1b, and GT1b) on the neuronal membrane. Thus, c mediates binding of TeNT to the presynaptic membrane of peripheral motor axons and aids movement of TeNT across that membrane into the neuron.

An amino acid sequence for:

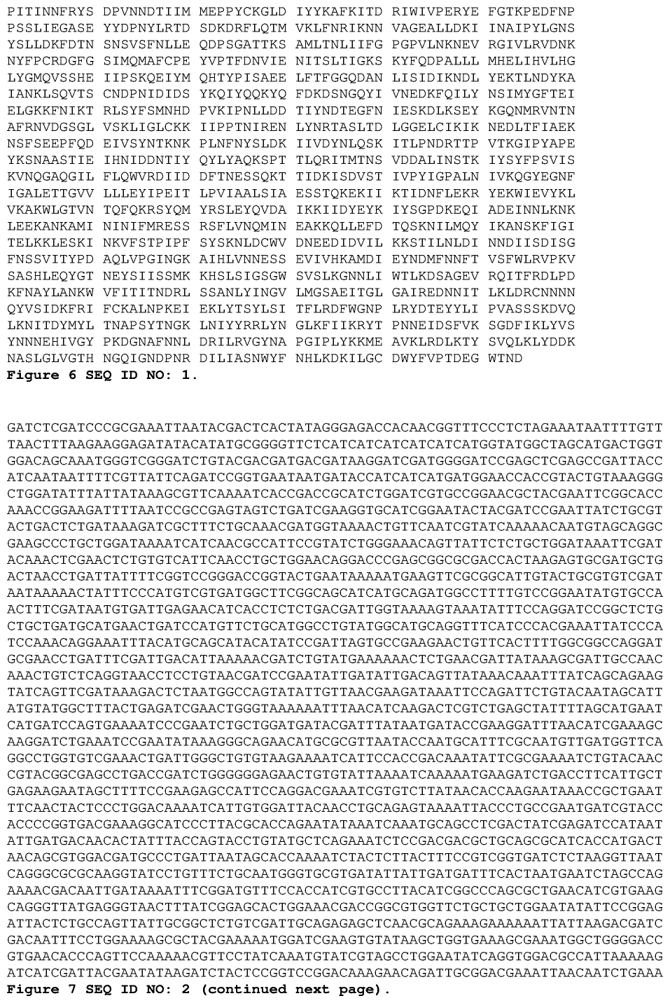

TeNT lacking the initiator methionine is provided in FIG. 6 (SEQ ID NO: 1);

HC is provided in FIG. 9 (SEQ ID NO: 3);

c is provided in FIG. 10 (SEQ ID NO: 4); and

A codon-optimised nucleic acid sequence of a vector encoding and a vector map for:

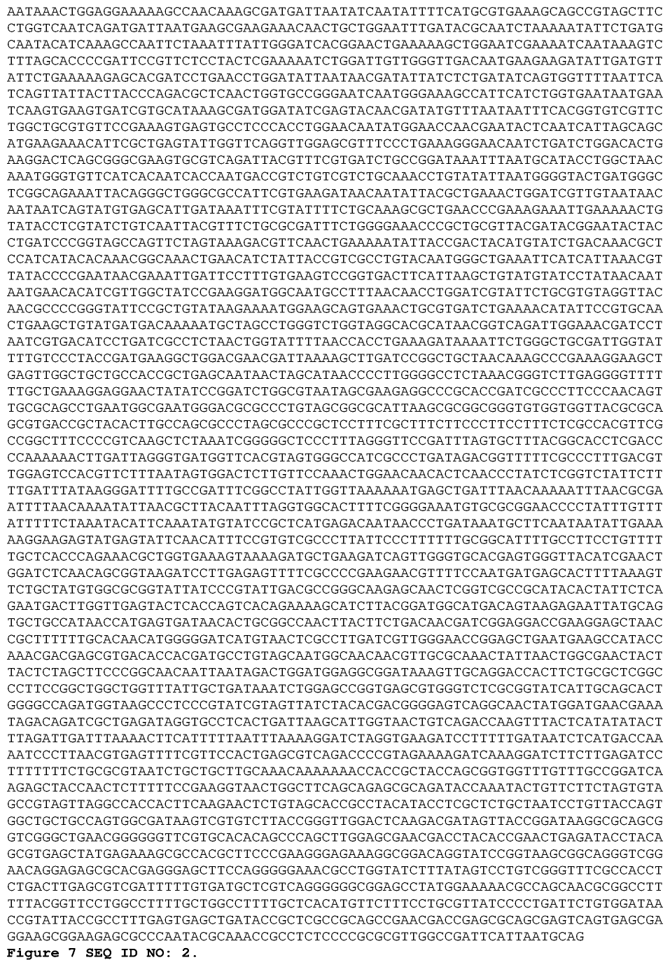

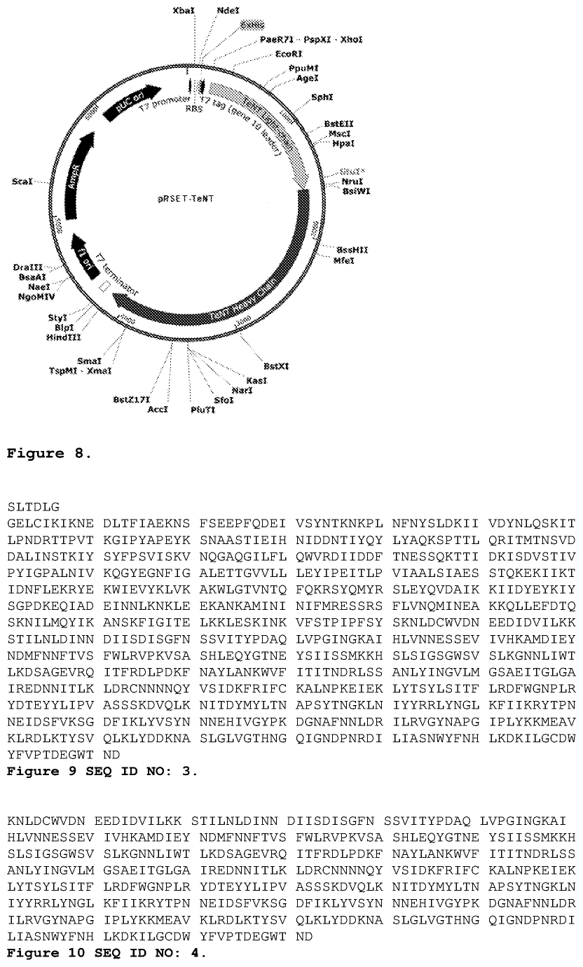

TeNT lacking the initiator methionine is provided in FIG. 7 (SEQ ID NO: 2) and FIG. 8; As used herein, “TeNT” is used in reference to a full TeNT molecule, consisting of the heavy chain and light chain. Subdomains and fragments are referred to herein by their abbreviations: light chain “LC”; heavy chain “HC”; heavy chain N-terminus domain “HN”; heavy chain fragment c “c”; light chain plus heavy chain N-terminus domain “LC-HN” (i.e. TeNT molecule lacking c). Where any subdomain or fragment is PEGylated, the prefix PEG is used: PEG-LC; PEG-HC; PEG-HN; PEG-c; PEG-LC-HN. In a full TeNT molecule comprising a PEGylated fragment or subdomain, the prefix PEG is used and the PEGylated subdomain or fragment is indicated: PEG-TeNT-LC; PEG-TeNT-HC; PEG-TeNT-LC-HC; PEG-TeNT-HN; PEG-TeNT-c; PEG-TeNT-LC-c; PEG-TeNT-LC-HN, and so on.

In a full TeNT molecule or subdomain comprising a masking polypeptide, the prefix PEP is used and the PEPylated subdomain or fragment is indicated: PEP-TeNT-LC; PEP-TeNT-HC; PEP-TeNT-LC-HC; PEP-TeNT-HN; PEP-TeNT-c; PEP-TeNT-LC-c; PEP-TeNT-LC-HN, and so on.

It will be appreciated that because of their specific functions, subdomains and fragments are not interchangeable for full TeNT.

TeNTs disclosed herein may be active or inactive. An active TeNT possesses the same biological activities of native TeNT. An inactive TeNT lacks one or more activities of native TeNT. In one embodiment, an inactive TeNT does not block release of inhibitory neurotransmitters. An inactive TeNT includes tetanus toxoid. In one embodiment, an inactive TeNT is an inactive TeNT as disclosed herein. The inactive TeNT may act as a decoy for the adaptive immune system, improving the activity of an active TeNT of the disclosure.

In one embodiment, two or more TeNTs may be conjugated.

In another embodiment, TeNTs are not conjugated. In one embodiment of the compositions, methods and uses disclosed herein, a first PEG-TeNT is not conjugated to a second TeNT.

Whilst not wishing to be bound by theory, the inventors consider that there is at least one distinct and important advantage in the TeNTs of the disclosure not being conjugated. Specifically, in one embodiment, in use, the composition provides one active TeNT that is masked by PEGylation or a peptide from the adaptive immune response of a subject in combination with in an inactive, unmasked decoy TeNT that simultaneously attracts the adaptive immune response. If these TeNTs were conjugated, the adaptive immune response would inactivate the conjugate by virtue of the unmasked decoy TeNT, despite the presence of the masked active TeNT. Therefore, in one embodiment, ensuring that the two TeNTs are not conjugated ensures that the composition is active.

In other words, in one embodiment, where two functions are desired, the functions are intended to be independent, and therefore the TeNTs of the invention are not conjugated.

Whilst not wishing to be bound by theory, the inventors also consider that conjugating two TeNTs may impair activity of the active TeNT or active TeNTs, possibly by provoking an immune response based on the size of the conjugate.

Despite these perceived advantages, in one embodiment, two or more TeNTs may be conjugated to one another.

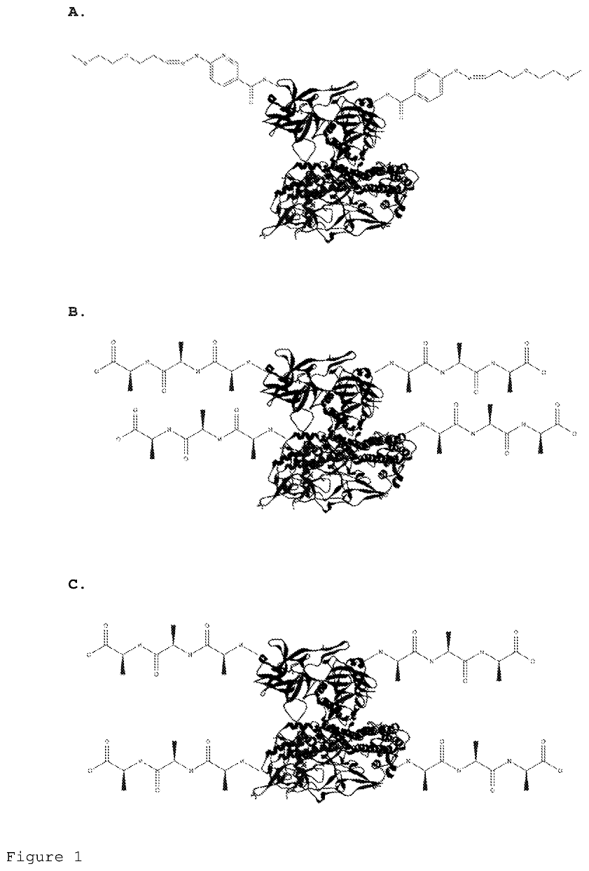

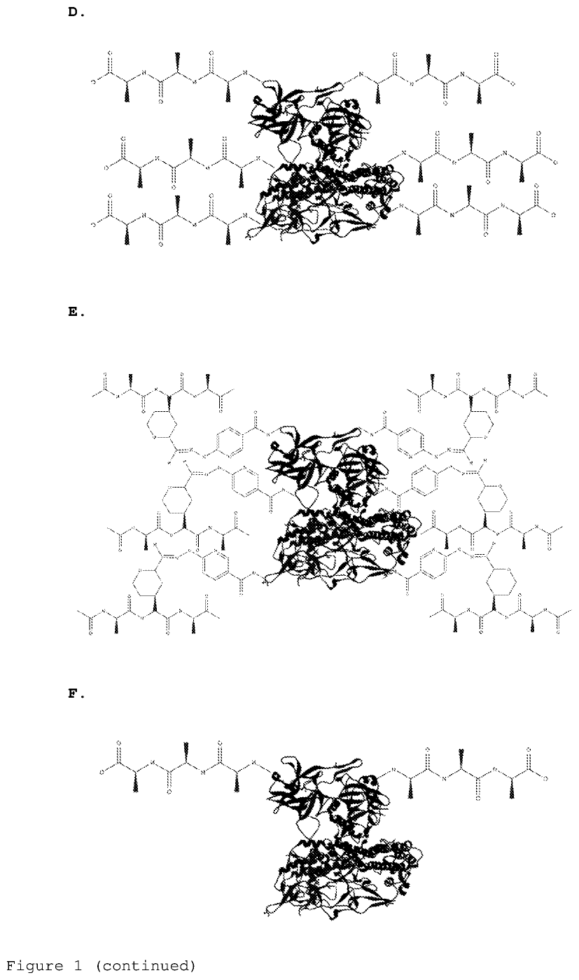

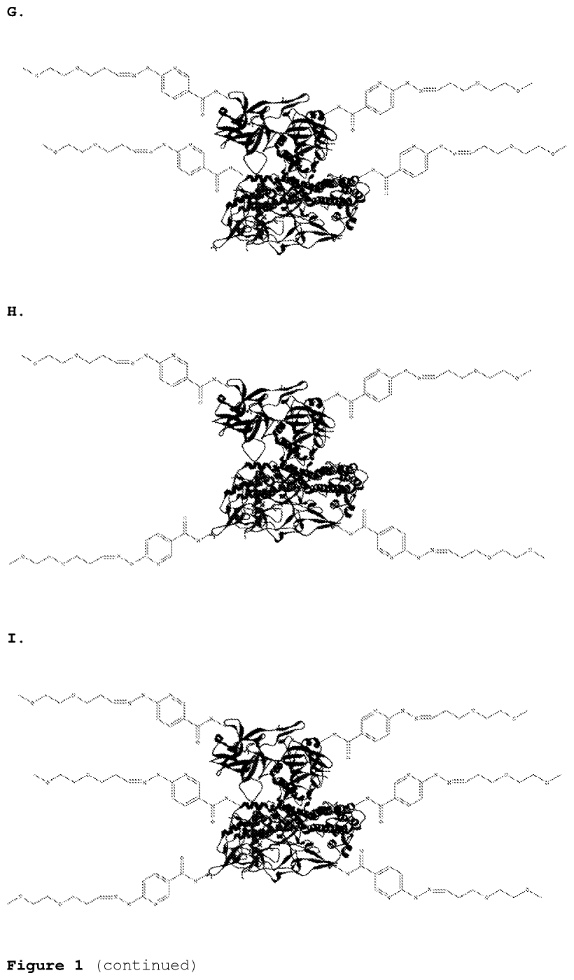

Immune-evading PEG-TeNTs and PEP-TeNTs disclosed herein and depicted in FIG. 1 include:

TeNT with hydrazone PEGylated fragment c (PEG-HZN-TeNT-c) (FIG. 1A) (Example 1);

TeNT with PEPylated heavy chain (PEP-TeNT-HC) (FIG. 1B) (Example 9);

TeNT with PEPylated light chain and fragment c (PEP-TeNT-LC-c (FIG. 1C) (Example 10);

TeNT with PEPylated light and heavy chains (PEP-TeNT-LC-HC) (FIG. 1D) (Example 11);

TeNT with Hydrazone PEPylated light and heavy chains (PEP-HZN-TeNT-LC-HC) (FIG. 1E) (Example 11);

TeNT with PEPylated fragment c (PEP-TeNT-c) (FIG. 1F) (Example 8);

TeNT with hydrazone PEGylated heavy chain (PEG-HZN-TeNT-HC) (FIG. 1G) (Example 2);

TeNT with hydrazone PEGylated light chain and PEGylated fragment c (PEG-HZN-TeNT-LC-c) (FIG. 1H) (Example 3); and

TeNT fully hydrazone PEGylated (PEG-HZN-TeNT-LC-HC) (FIG. 1I) (Example 5).

PEG-HZN-TeNT-c advantageously evades the pre-existing immune response of the adaptive immune system in vaccinated subjects. In one embodiment, PEG-TeNT-c provides first tier treatment to be used until efficacy decreases.

PEG-TeNT-HC and PEG-TeNT-LC-c advantageously evade the pre-existing immune response of the adaptive immune system in vaccinated subjects and also evade the immune response of the adaptive immune system elicited by repeated exposure to PEG-TeNT-c.

In one embodiment, PEG-TeNT-HC and PEG-TeNT-LC-c together provide second tier treatment to be used until their efficacy decreases.

PEG-TeNT-LC-HC advantageously evades the pre-existing immune response of the adaptive immune system in vaccinated subjects and also evades the immune response of the adaptive immune system elicited by repeated exposure to PEG-TeNT-c and to PEG-TeNT-HC plus PEG-TeNT-LC-c.

In one embodiment, PEG-TeNT-LC-HC provides third tier treatment to be used until its efficacy decreases.

Also disclosed is TeNT with PEGylated light chain (PEG-TeNT-LC), TeNT with PEGylated LC and HN (PEG-TeNT-LC-HN), and TeNT with PEGylated HN (PEG-TeNT-HN).

The person skilled in the art will appreciate that the specific combinations of PEG-TeNTs and the order of treatment with those PEG-TeNT combinations may be altered.

### Polyethylene Glycol (PEG)

PEG may be conjugated via an acid labile linkage to, for example, lysine (e.g. amino-PEGylation), cysteine (e.g. thiol-PEGylation and bridging PEGylation), histidine, arginine, aspartic acid, asparagine (e.g. N-glyco-PEGylation), glutamic acid, glutamine (e.g. transglutaminase-mediated PEGylation), serine (e.g. O-glyco-PEGylation), threonine (e.g. O-glyco-PEGylation), or tyrosine residues in TeNT. Examples of PEGylation also include N-terminus PEGylation and C-terminus PEGylation.

PEGylation may be achieved by reacting PEG with a functional group that is hydroxyl-reactive, for example anhydrides, acid chlorides, chloroformates and carbonates. Alternatively, PEGylation may be achieved with functional groups such as aldehyde, ester, and amide.

PEG may be linear or branched.

PEG may be a modified PEG, for example, poly[oligo(ethylene glycol) methyl ether methacrylate] (POEGMA).

PEGylation may be site-specific PEGylation.

In one embodiment, surface serine residues of TeNT or a TeNT fragment are mutated to surface cysteine residues to facilitate directed PEG conjugation at immunogenic epitopes. In this context, a mutation is synonymous with substitution, for instance, a serine to cysteine substitution. Such mutations, or substitutions, include one or more, in any combination, of: S81C; S120C; S144C; S248C; S335C; S428C; S600C; S963C; S1041C; S1155C; and S1187C.

Functional groups for heterobifunctional PEGs include maleimide, vinyl sulfone, pyridyl disulfide, amine, carboxylic acid, and NHS ester.

In one embodiment, PEG is conjugated to a TeNT using carboxyl-to-amine crosslinking using the carbodiimide-EDC and sulfo-NHS.

The invention also contemplates a PEG-TeNT comprising different molecular weight PEGs conjugated to different subdomains or fragments of TeNT.

PEG may be conjugated or attached to TeNTs of the disclosure between 4° C. and 25° C. for between 2 and 6 hours, for example. In one embodiment, PEG was conjugated to TeNT at room temperature for 6 hours.

### Making Polypeptide (PEP)

Masking polypeptide may be conjugated, for example, to lysine (e.g. amino-PEPylation), cysteine (e.g. thiol-PEPylation and bridging PEPylation), histidine, arginine, aspartic acid, asparagine (e.g. N-glyco-PEPylation), glutamic acid, glutamine (e.g. transglutaminase-mediated PEPylation), serine (e.g. O-glyco-PEPylation), threonine (e.g. O-glyco-PEPylation), or tyrosine residues in TeNT. Examples of PEPylation also include N-terminus PEPylation and C-terminus PEPylation.

PEPylation may be achieved by reacting PEP with a functional group that is hydroxyl-reactive, for example anhydrides, acid chlorides, chloroformates and carbonates. Alternatively, PEGylation may be achieved with functional groups such as aldehyde, ester, and amide.

PEP may be linear or branched.

PEP may be a repeating or random sequences of glycine and threonine (GT-PEP) with or without O-linked glycosylation.

PEPylation may be site-specific PEPylation.

In one embodiment, surface serine residues of TeNT or a TeNT fragment are mutated to surface cysteine residues to facilitate directed PEP conjugation at immunogenic epitopes. In this context, a mutation is synonymous with substitution, for instance, a serine to cysteine substitution. Such mutations, or substitutions, include one or more, in any combination, of: S81C; S120C; S144C; S248C; S335C; S428C; S600C; S963C; S1041C; S1155C; and S1187C.

Functional groups for heterobifunctional PEPs include maleimide, vinyl sulfone, pyridyl disulfide, amine, carboxylic acid, and NHS ester.

In one embodiment, PEP is conjugated to a TeNT using carboxyl-to-amine crosslinking using the carbodiimide-EDC and sulfo-NHS.

The invention also contemplates a PEP-TeNT comprising different molecular weight PEPs conjugated to different subdomains or fragments of TeNT.

PEP may be conjugated or attached to TeNTs of the disclosure between 4° C. and 25° C. for between 2 and 6 hours, for example. In one embodiment, PEP was conjugated to TeNT at room temperature for 6 hours.

Without wishing to be bound by theory, the inventors contemplate that PEP may be less immunogenic than PEG, and may be particularly useful in subjects allergic to PEG, and PEP may be less detrimental to TeNT activity than PEG by maintaining solubility of a TeNT near endogenous solubility of TeNT, and/or minimizing membrane interactions, for example.

### Hydrazone Linkage

Hydrazone linkage may be introduced between PEG molecules and TeNT or between masking polypeptides and TeNT where PEG or polypeptides may be conjugated, for example, to lysine (e.g. amino-PEGylation, amino-PEPylation), cysteine (e.g. thiol-PEGylation and bridging PEGylation, thiol-PEGylation and bridging PEGylation), histidine, arginine, aspartic acid, asparagine (e.g. N-glyco-PEGylation), glutamic acid, glutamine (e.g. transglutaminase-mediated PEGylation), serine (e.g. O-glyco-PEGylation), threonine (e.g. O-glyco-PEGylation), or tyrosine residues in TeNT. Hydrazone may be introduced by reacting a hetero-bifunctional cross-linker, such as Succinimidyl 6-hydrazinonicotinamide acetone hydrazine (SANH), Succinimidyl 6-hydraziniumnicotinate hydrochloride (SHNH), N-(β-maleimidopropionic acid) hydrazide (BMPH), N-ε-maleimidocaproic acid hydrazide (EMCH) or N-κ-maleimidoundecanoic acid hydrazide (KMUH), with surface lysines or cysteines on TeNT and aldehyde groups on PEG or Peptides. Hydrazone may be introduced by chemical methods by introducing a nicotinamide group onto surface lysines or cysteines on TeNT and linking to carbonyl groups on PEG or Peptides. Examples of PEGylation also include N-terminus PEGylation and C-terminus PEGylation. Hydrazone may be introduced by reacting hydrazide with the carbonyl group of oxidised carbohydrate on a glycol-peptide followed by cross-linking to TeNT with a heterobifunctional cross linker to cysteine residues.

### Glycopeptides

Peptides may be produced as glycopeptides by expression in eukaryotic host systems where carbohydrates are attached to the peptide by N-linked glycosylation or O-linked glycosylation. Glycopeptides may be conjugated directly to TeNT as masking agents for example, to lysine (e.g. amino-PEGylation, amino-PEPylation), cysteine (e.g. thiol-PEGylation and bridging PEGylation, thiol-PEGylation and bridging PEGylation), histidine, arginine, aspartic acid, asparagine (e.g. N-glyco-PEGylation), glutamic acid, glutamine (e.g. transglutaminase-mediated PEGylation), serine (e.g. O-glyco-PEGylation), threonine (e.g. O-glyco-PEGylation), or tyrosine residues in TeNT. Examples of PEGylation also include N-terminus PEGylation and C-terminus PEGylation. The carbohydrate residues of the glycoprotein may be oxidised to introduce carbonyl groups which may be used to conjugate the glycopeptides to TeNT directly or to introduce pH-labile linkers between the glycopeptides and TeNT.

### Indications

As used herein, “hypotonia” refers to any disorder comprising involuntary muscle weakness that may be treated by inhibiting inhibitory neurotransmitters, for instance GABA or glycine. As such, “hypotonia” includes reduced muscle tone secondary to reduced neurological drive or other causes and conditions of reduced or inadequate muscle tone, strength or neurological drive. Thus, in one embodiment, hypotonia may be neurological hypotonia. In one embodiment, hyotonia may be sleep-induced, i.e. a muscle may be hypotonic during sleep relative to the same muscle during waking periods.

Hypotonia disorders that may be treated with a TeNT conjugate, a composition or method according to the disclosure include obstructive sleep apnoea, apnoea, snoring, ptosis, Horner's syndrome, muscle atrophy, neurologically impaired muscles, amyotrophic lateral sclerosis (ALS), motor neuron disease, any myopathy, multiple sclerosis, myasthenia gravis, decrease in facial muscle tone, optionally ectropion, flaccid paralysis or weakness of any cause of any skeletal or smooth muscle, respiratory muscle weakness of any cause, including post-ventilator weakness, trauma-induced muscle weakness or poor posture caused by muscular flaccidity, pelvic floor muscle flaccidity or weakness, or nasal or upper respiratory flaccidity.

Other disorders that may be treated with a TeNT conjugate, a composition or method according to the disclosure include muscular atrophy, or decrease in muscle mass.

In the event that a disorder to be treated according to the disclosure is not a hypotonia disorder per se, increasing muscle tone by treating with a TeNT of the disclosure may alleviate a symptom of the disorder.

Cosmetic applications of TeNT conjugates described herein may include tightening of the abdominal muscles, tightening of the pectoral muscles, tightening of the gluteus maximus, tightening of skeletal muscles, or treatment of facial droop caused by muscle flaccidity.

Smooth muscles, skeletal muscles, tissues or organs that may be treated with a TeNT conjugate of the disclosure, a composition or method according to the disclosure include upper oesophagus, oesophageal wall, oesophageal sphincter, lower oesophageal sphincter, anal sphincter, bladder, bladder sphincter, vaginal sphincter, pyloric sphincter, sphincter of Oddi, ileocaecal sphincter, pelvic floor muscles, prostate gland, submandibular gland, parotid gland, sublingual gland, minor salivary glands of the oral mucosa, vocal folds, vocal cords, laryngeal muscles, facial muscles, chin muscles, chin lifting muscles, mastication muscles, scalp muscles, chest muscles, back muscles, upper limb muscles, forearm muscles, lower limb muscles, hand muscles, foot muscles, stomach wall muscles, colon wall muscles, neck muscles, throat dilator muscles, masseter muscle, medial ptygeroid, lateral ptygeroid, geniohyoid, genioglossus, tensor veli palatine, levator veli palatini, stylopharyngeus, styloglossus, mylohyoid, stylohyoid, hyoglossus, diagastricus, sternocleidomastoid muscle, trapezius muscle, temporalis muscles, cricopharyngeus muscle, uterine muscle and cervix, gastric nerve supply, intranasal mucosa, pulmonary mucosa, skin, thymus, bone, coronary artery, pulmonary smooth muscle, and cardiac muscle.

In one embodiment, laryngeal hemiplegia, recurrent laryngeal neuropathy or Roarer Syndrome may be treated with a TeNT conjugate of the disclosure. These indications may be treated in an equine or canine subject, particularly a horse or a dog.

The TeNT conjugates described herein may also be used to enhance muscle tone, muscle power, muscle healing and/or sporting performance.

### Compositions and Administration

The composition of the disclosure may be a therapeutic composition or a cosmetic composition. That is, the composition may be used for therapy or cosmetic purposes.

As used herein, the term “therapeutic composition” or “cosmetic composition” refers to a composition comprising a TeNT that inhibits or treats hypotonia in the subject as described herein. The composition has been formulated for administration to a subject. In one embodiment, the composition is sterile. In one embodiment, the composition is pyrogen-free. The composition may comprise a pharmaceutically acceptable carrier. Preferably, the composition is manufactured according to Good Laboratory Practice (GLP) or Good Manufacturing Practice (GMP).

A TeNT conjugate of the disclosure may be administered at up to 10 mg/kg or more. A TeNT conjugate of the disclosure may be administered at about 1 fg/kg, about 5 fg/kg, about 10 fg/kg, about 50 fg/kg, about 100 fg/kg, about 500 fg/kg, about 1 pg/kg, about 5 pg/kg, about 10 pg/kg, about 50 pg/kg, about 100 pg/kg, about 500 pg/kg, about 1 ng/kg, about 2 ng/kg, about 3 ng/kg, about 4 ng/kg, about 5 ng/kg, about 6 ng/kg, about 7 ng/kg, about 8 ng/kg, about 9 ng/kg, about 10 ng/kg, about 11 ng/kg, about 12 ng/kg, about 13 ng/kg, about 14 ng/kg, about 15 ng/kg, about 16 ng/kg, about 17 ng/kg, about 18 ng/kg, about 19 ng/kg, about 20 ng/kg, about 30 ng/kg, about 40 ng/kg, about 50 ng/kg, about 60 ng/kg, about 70 ng/kg, about 80 ng/kg, about 90 ng/kg, about 100 ng/kg, about 200 ng/kg, about 300 ng/kg, about 400 ng/kg, about 500 ng/kg, about 600 ng/kg, about 700 ng/kg, about 800 ng/kg, about 900 ng/kg, about 1 μg/kg, about 5 μg/kg, about 10 μg/kg, about 50 μg/kg, about 100 μg/kg, about 500 μg/kg, about 1 mg/kg, or about 10 mg/kg. A TeNT conjugate of the disclosure may be administered within any range of any of the doses listed above.

A TeNT conjugate of the disclosure may be administered at up to 1000 IU/kg or more. A TeNT conjugate of the disclosure may be administered at about 0.1 IU/kg, about 0.2 IU/kg, about 0.3 IU/kg, about 0.4 IU/kg, about 0.5 IU/kg, about 0.6 IU/kg, about 0.7 IU/kg, about 0.8 IU/kg, about 0.9 IU/kg, about 1 IU/kg, about 2 IU/kg, about 3 IU/kg, about 4 IU/kg, about 5 IU/kg, about 6 IU/kg, about 7 IU/kg, about 8 IU/kg, about 9 IU/kg, about 10 IU/kg, about 11 IU/kg, about 12 IU/kg, about 13 IU/kg, about 14 IU/kg, about 15 IU/kg, about 16 IU/kg, about 17 IU/kg, about 18 IU/kg, about 19 IU/kg, about 20 IU/kg, about 30 IU/kg, about 40 IU/kg, about 50 IU/kg, about 60 IU/kg, about 70 IU/kg, about 80 IU/kg, about 90 IU/kg, about 100 IU/kg, about 200 IU/kg, about 300 IU/kg, about 400 IU/kg, about 500 IU/kg, about 600 IU/kg, about 700 IU/kg, about 800 IU/kg, about 900 IU/kg, about 1 000 IU/kg. A TeNT conjugate of the disclosure may be administered within any range of any of the doses listed above.

In a composition comprising two TeNT conjugates, for example a composition comprising a first TeNT conjugate, wherein TeNT-HC is PEGylated (PEG-TeNT-HC), and a second TeNT conjugate wherein TeNT-LC is PEGylated and TeNT-c is PEGylated (PEG-TeNT-LC-c), the ratio of the first TeNT conjugate to the second TeNT conjugate may be varied. For example, the ratio of first TeNT conjugate to second TeNT conjugate may be about 1000:1, about 500:1, about 100:1, about 50:1, about 10:1, about 5:1, about 4:1, about 3:1, about 2:1, about 1:1; about 1:2, about 1:3, about 1:4, about 1:5, about 1:10, about 1:50, about 1:100, about 1:500, or about 1:1000.

A composition may comprise any combination of TeNT conjugates and is not to be limited to a combination of PEG-TeNT-HC and PEG-TeNT-LC-c. A composition may comprise: PEG-TeNT-c and PEG-TeNT-HC; PEG-TeNT-c and PEG-TeNT-LC-c; PEG-TeNT-c and PEG-TeNT-LC-HC; PEG-TeNT-HC and PEG-TeNT-LC-HC; and PEG-TeNT-LC-c and PEG-TeNT-LC-HC. Also disclosed is a composition comprising: PEG-TeNT-c, PEG-TeNT-HC and PEG-TeNT-LC-c; PEG-TeNT-c, PEG-TeNT-HC and PEG-TeNT-LC-HC; PEG-TeNT-c, PEG-TeNT-LC-c and PEG-TeNT-LC-HC; PEG-TeNT-HC, PEG-TeNT-LC-c and PEG-TeNT-LC-HC; and PEG-TeNT-c, PEG-TeNT-HC, PEG-TeNT-LC-c and PEG-TeNT-LC-HC. In a composition, any TeNT may be substituted for, and any composition may further comprise, PEG-TeNT-LC, PEG-TeNT-LC-HN, and/or PEG-TeNT-HN.

A composition may comprise: PEP-TeNT-c and PEP-TeNT-HC; PEP-TeNT-c and PEP-TeNT-LC-c; PEP-TeNT-c and PEP-TeNT-LC-HC; PEP-TeNT-HC and PEP-TeNT-LC-HC; and PEP-TeNT-LC-c and PEP-TeNT-LC-HC. Also disclosed is a composition comprising: PEP-TeNT-c, PEP-TeNT-HC and PEP-TeNT-LC-c; PEP-TeNT-c, PEP-TeNT-HC and PEP-TeNT-LC-HC; PEP-TeNT-c, PEP-TeNT-LC-c and PEP-TeNT-LC-HC; PEP-TeNT-HC, PEP-TeNT-LC-c and PEP-TeNT-LC-HC; and PEP-TeNT-c, PEP-TeNT-HC, PEP-TeNT-LC-c and PEP-TeNT-LC-HC.

A TeNT conjugate or composition of the disclosure may be administered once, twice or three times per week, once, twice or three times per month, once, twice or three times per quarter, once, twice or three times per 6 months, or once, twice or three times per year.

The TeNT conjugate or composition may be administered in a single dose, a split dose, or in multiple doses. Where a muscle exists as a pair, the PEG-TeNT may be administered unilaterally to one muscle of the pair or bilaterally to both muscles of the pair.

As an alternative to a composition comprising two or more PEG-TeNTs of the disclosure, such two or more TeNT conjugates or compositions thereof may be administered in combination sequentially or simultaneously.

The TeNT conjugate or composition may be administered to a subject locally by any suitable method, for example by injection, surgical implantation, topical application, or intranasal administration. In one embodiment, the TeNT conjugate is administered intramuscularly by injection to the affected muscle.

The TeNT conjugate or composition will be formulated, dosed, and administered in a fashion consistent with good medical practice. Factors for consideration in this context include the particular type of hypotonia being treated, the particular subject being treated, the clinical condition of the subject, the site of administration, the method of administration, the scheduling of administration, and other factors known to medical, including dental, practitioners. The therapeutically effective amount of the TeNT conjugate to be administered will be governed by such considerations.

The TeNT conjugate or composition may be formulated in a sustained release formulation as known to the person skilled in the art.

Pharmaceutically acceptable carriers include water, buffered water, saline solutions such as, for example, normal saline or balanced saline solutions such as Hank's or Earle's balanced solutions, glycine, and hyaluronic acid.

The TeNT conjugate or composition may be formulated for intramuscular administration. Compositions for intramuscular administration may comprise pharmaceutically acceptable sterile aqueous or non-aqueous solutions, dispersions, suspensions or emulsions as well as sterile powders for reconstitution into sterile injectable solutions or dispersions. Examples of suitable aqueous and non-aqueous carriers, solvents, diluents or vehicles include water, ethanol, polyols (such as glycerol, propylene glycol, polyethylene glycol), carboxymethylcellulose and mixtures thereof, vegetable oils (such as olive oil), injectable organic esters (e.g. ethyl oleate).

The composition may comprise penetration enhancers to enhance their delivery of TeNT. Penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di-glycerides and physiologically acceptable salts thereof.

The composition may further include chelating agents such as, for example, ethylenediaminetetraacetic acid (EDTA), citric acid, salicylates (e.g. sodium salycilate, 5-methoxysalicylate, homovanilate).

Also provided is an article of manufacture and/or a kit, comprising a container comprising the PEG-TeNT or composition comprising the TeNT conjugate. The container may be a bottle, vial or syringe comprising the TeNT conjugate or composition, optionally in unit dosage form. For example, the TeNT conjugate or composition may be in the form of an injectable solution in a disposable container, optionally a syringe. The article of manufacture and/or kit may further comprise printed instructions and/or a label or the like, indicating treatment of a subject according to the method disclosed herein.

The term “therapeutically effective amount” refers to an amount of TeNT conjugate effective to treat hypotonia in a subject.

The terms “treat”, “treating” or “treatment” refer to both therapeutic treatment and prophylactic or preventative measures, wherein the aim is to prevent, reduce, or ameliorate hypotonia in a subject or slow down (lessen) progression of hypotonia in a subject. Subjects in need of treatment include those already with hypotonia as well as those in which hypotonia is to be prevented or ameliorated.

The terms “preventing”, “prevention”, “preventative” or “prophylactic” refers to keeping from occurring, or to hinder, defend from, or protect from the occurrence of hypotonia. A subject in need of prevention may be prone to develop hypotonia.

The term “ameliorate” or “amelioration” refers to a decrease, reduction or elimination of hypotonia.

Hypotonia may be quantified. Hypotonia may be quantified on a semi-quantitative scale, for example 0 to 5, where 0 represents absence, 1 to 4 represent identifiable increases in severity, and 5 represents maximum severity. Alternatively, hypotonia may be quantified as a binary event, i.e. presence or absence, 0 or 1. Other semi-quantitative scales will be readily apparent to the person skilled in the art. In another embodiment, hypotonia may be quantified on a quantitative scale, for instance using a force gauge.

Any quantification of hypotonia may be compared to a control, for example a healthy control subject not receiving a PEG-TeNT, an affected control subject receiving treatment for hypotonia, but not treated with a TeNT conjugate, or a population.

Treating hypotonia by administering a TeNT conjugate may be about a 1% decrease, about a 2% decrease, about a 3% decrease, about a 4% decrease, about a 5% decrease, about a 6% decrease, about a 7% decrease, about an 8% decrease, about a 9% decrease, about a 10% decrease, about a 20% decrease, about a 30% decrease, about a 40% decrease, about a 50% decrease, about a 60% decrease, about a 70% decrease, about an 80% decrease, about a 90% decrease, or about a 100% decrease in the hypotonia.

As used herein, the term “subject” may refer to a mammal. The mammal may be a primate, particularly a human, or may be a domestic, zoo, or companion animal. Although it is particularly contemplated that the PEG-TeNTs, compositions and method disclosed herein are suitable for medical treatment of humans, it is also applicable to veterinary treatment, including treatment of domestic animals such as horses, cattle and sheep, companion animals such as dogs and cats, or zoo animals such as felids, canids, bovids and ungulates.

Unless defined otherwise in this specification, technical and scientific terms used herein have the same meaning as commonly understood by the person skilled in the art to which this invention belongs and by reference to published texts.

It is to be noted that the term “a” or “an” refers to one or more, for example, “a TeNT” is understood to represent one or more TeNTs. As such, the terms “a” or “an”, “one or more,” and “at least one” may be used interchangeably herein.

In the claims which follow and in the description of the invention, except where the context requires otherwise due to express language or necessary implication, the word “comprise” or variations such as “comprises” or “comprising” is used in an inclusive sense, i.e. to specify the presence of the stated features, but not to preclude the presence or addition of further features in various embodiments of the invention.

The term “about” as used herein contemplates a range of values for a given number of ±25% the magnitude of that number. In other embodiments, the term “about” contemplates a range of values for a given number of ±20%, ±15%, ±10%, ±5%, ±4%, ±3%, ±2%, or ±1% the magnitude of that number. For example, in one embodiment, “about 3 grams” indicates a value of 2.7 grams to 3.3 grams (i.e. 3 grams±10%), and the like.

Similarly, the timing or duration of events may be varied by at least 25%. For example, while a particular event may be disclosed in one embodiment as lasting one day, the event may last for more or less than one day. For example, “one day” may include a period of about 18 hours to about 30 hours. In other embodiments, periods of time may vary by ±20%, ±15%, ±10%, ±5%, ±4%, ±3%, ±2%, or ±1% of that period of time.

### EXAMPLES

### Example 1—Preparation of PEG-HZN-TeNT-c (FIG. 1A)

In this example, surface serine residues of TeNT-c will be mutated to surface cysteine residues to facilitate directed PEG conjugation at immunogenic epitopes to produce the molecule of FIG. 1A. The mutations will be: S963C, S1041C, S1155C, and S1187C.

The gene for TeNT with surface serine to cysteine substitutions S963C, S1041C, S1155C, and S1187C will be synthesised by a commercial provider, for example Integrated DNA Technologies. The gene will be sub-cloned into the pRSET-A expression vector by restriction digestion so that 6× Histidine tag from the vector is added to the N-terminus of the mutant protein.

TeNT comprising S963C, S1041C, S1155C, and S1187C will be expressed, PEGylated and purified according to Example 5 to produce PEG-TeNT-c.

### Example 2—Preparation of PEG-HZN-TeNT-HC (FIG. 1G)

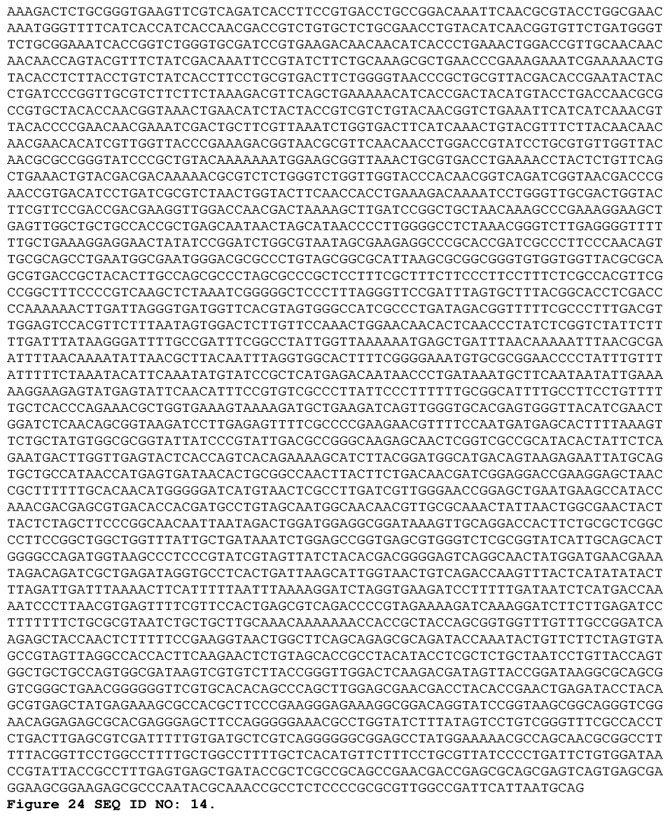

In this example, surface serine residues of TeNT-HC will be mutated to surface cysteine residues to facilitate directed PEG conjugation at immunogenic epitopes to produce the molecule of FIG. 1G. The mutations will be: S600C, S963C, S1041C, S1155C, and S1187C (FIG. 24 SEQ ID NO: 16).

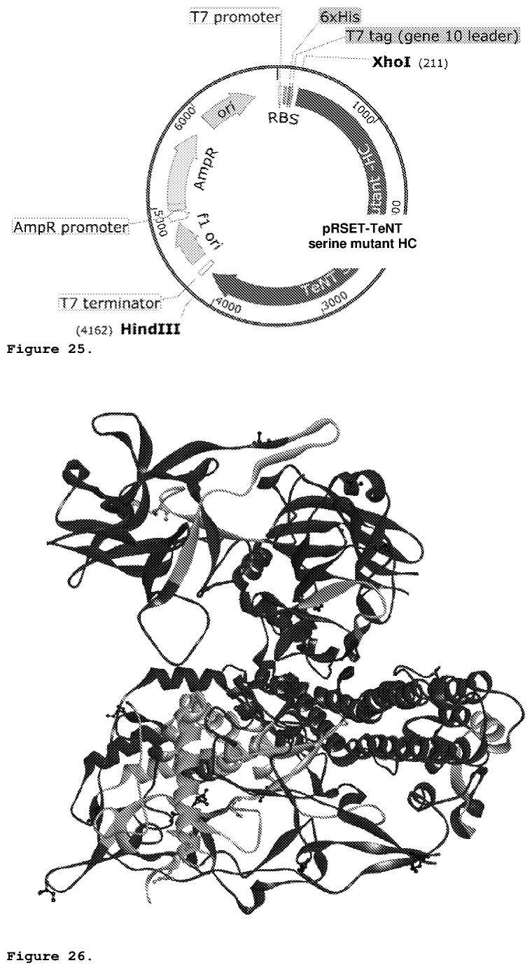

The gene for TeNT with surface serine to cysteine substitutions S600C, S963C, S1041C, S1155C, and S1187C will be synthesised by a commercial provider, for example Integrated DNA Technologies. The gene will be sub-cloned into the pRSET-A expression vector (FIG. 25 SEQ ID NO: 17, FIG. 26) by restriction digestion so that 6× Histidine tag from the vector is added to the N-terminus of the mutant protein.

TeNT comprising S600C, S963C, S1041C, S1155C, and S1187C will be expressed, PEGylated and purified according to Example 5 to produce PEG-TeNT-HC.

2.1—Endotoxin Removal

Endotoxin was removed from the TeNT-LC-c Serine mutant according to Example 4.

2.2—Generation of PEG-HZN-Maleimide

1. A molar excess of BMPH, EMCH or KMUH will be combined with PEG-Propionaldehyde, with PEG of about 2 kDa, about 5 kDa, about 10 kDa or about 20 kDa, in PBS (pH6.5-7.5) or 10-100% DMF, under normal atmosphere, a nitrogen atmosphere or an argon atmosphere.

2. Attachment will be performed between 4 and 37° C. for between 1 and 16 hours.

2.3—PEG Attachment to Cysteine Residues

1. A molar excess of PEG-HZN-maleimide, with PEG of about 2 kDa, about 5 kDa, about 10 kDa or about 20 kDa, was combined with serine mutant TeNT-LC-c (0.5-2 mg/mL) in PBS or <10% DMF at pH 6.5-7.5.

2. Attachment was performed at room temperature for 6 hours.

3. Excess PEG was removed by size exclusion chromatography.

2.4—Trypsin Digest Activation of Protein

Trypsin digestion for activation of the TeNT-LC-c Serine mutant was performed according to Example 4.

### Example 3—Preparation of PEG-HZN-TeNT-LC-c (FIG. 1H)

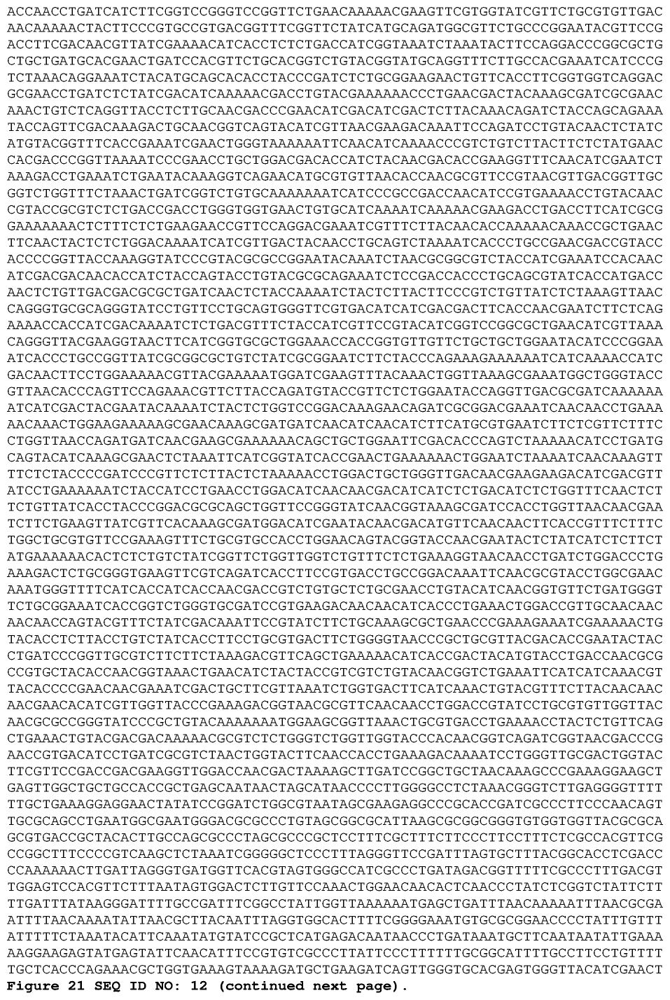

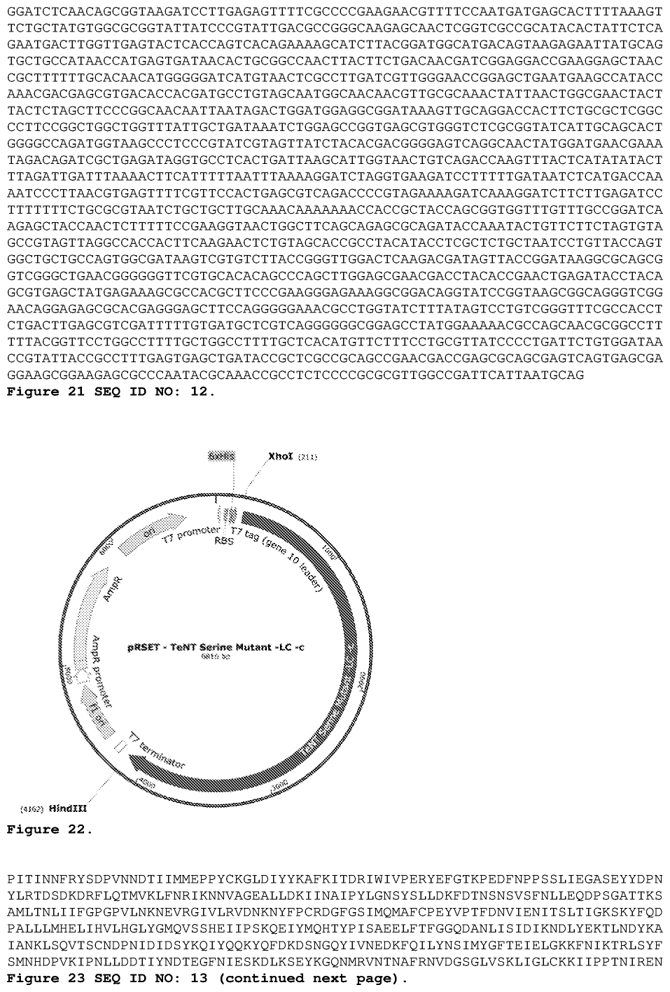

In this example, surface serine residues of LC and c will be mutated to surface cysteine residues to facilitate directed PEG conjugation at immunogenic epitopes to produce the molecule of FIG. 1H. The mutations were: S81C, S120C, S144C, S248C, S335C, S428C, S963C, S1041C, S1155C, and S1187C (FIG. 21 SEQ ID NO: 14).

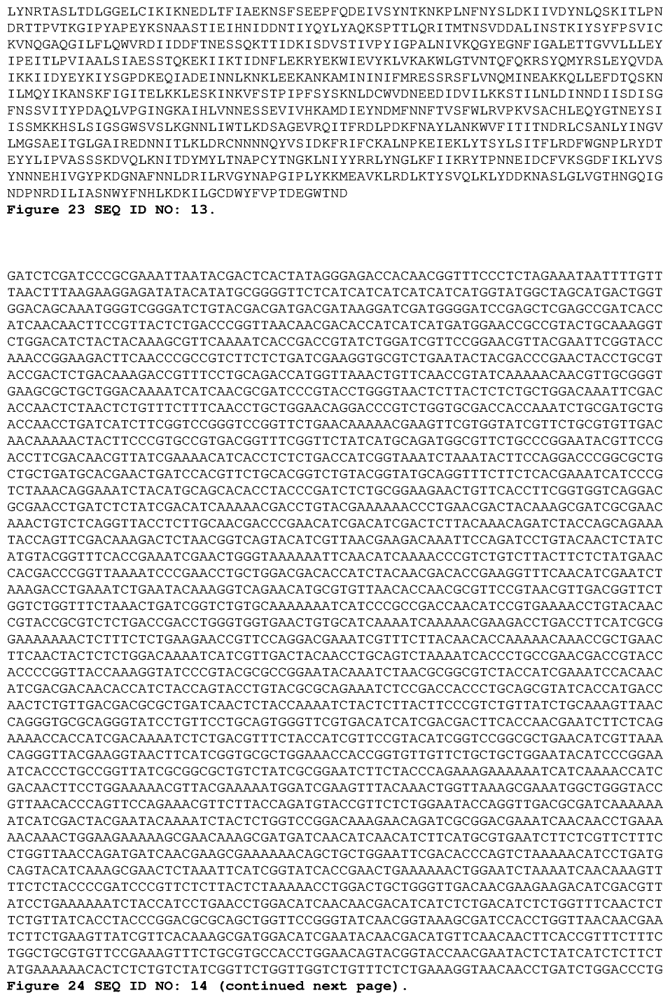

The gene for TeNT with surface serine to cysteine substitutions S81C, S120C, S144C, S248C, S335C, S428C, S963C, S1041C, S1155C, and S1187C in LC and c will be synthesised by a commercial provider, Integrated DNA Technologies. The gene will be sub-cloned into the pRSET-A expression vector (FIG. 22 SEQ ID NO: 15, FIG. 23) by restriction digestion so that 6× Histidine tag from the vector was added to the N-terminus of the mutant protein.

TeNT comprising S81C, S120C, S144C, S248C, S335C, S428C, S963C, S1041C, S1155C, and S1187C will be expressed, PEGylated and purified according to Example 5 to produce PEG-TeNT-LC-c.

3.1—Endotoxin Removal

Endotoxin will be removed from the TeNT-LC-c Serine mutant according to Example 4.

3.2—Generation of PEG-HZN-Maleimide

1. A molar excess of BMPH, EMCH or KMUH will be combined with PEG-Propionaldehyde, with PEG of about 2 kDa, about 5 kDa, about 10 kDa or about 20 kDa, in PBS (pH6.5-7.5) or 10-100% DMF, under normal atmosphere, a nitrogen atmosphere or an argon atmosphere.

2. Attachment will be performed between 4° C. and 37° C. for between 1 and 16 hours.

3.3—PEG Attachment to Cysteine Residues

1. A molar excess of PEG-HZN-maleimide, with PEG of about 2 kDa, about 5 kDa, about 10 kDa or about 20 kDa, will be combined with serine mutant TeNT-LC-c (0.5-2 mg/mL) in PBS or <10% DMF at pH 6.5-7.5.

2. Attachment will be performed at room temperature for 6 hours.

3. Excess PEG will be removed by size exclusion chromatography.

3.4—Trypsin Digest Activation of Protein

Trypsin digestion for activation of the TeNT-LC-c Serine mutant will be performed according to Example 4.

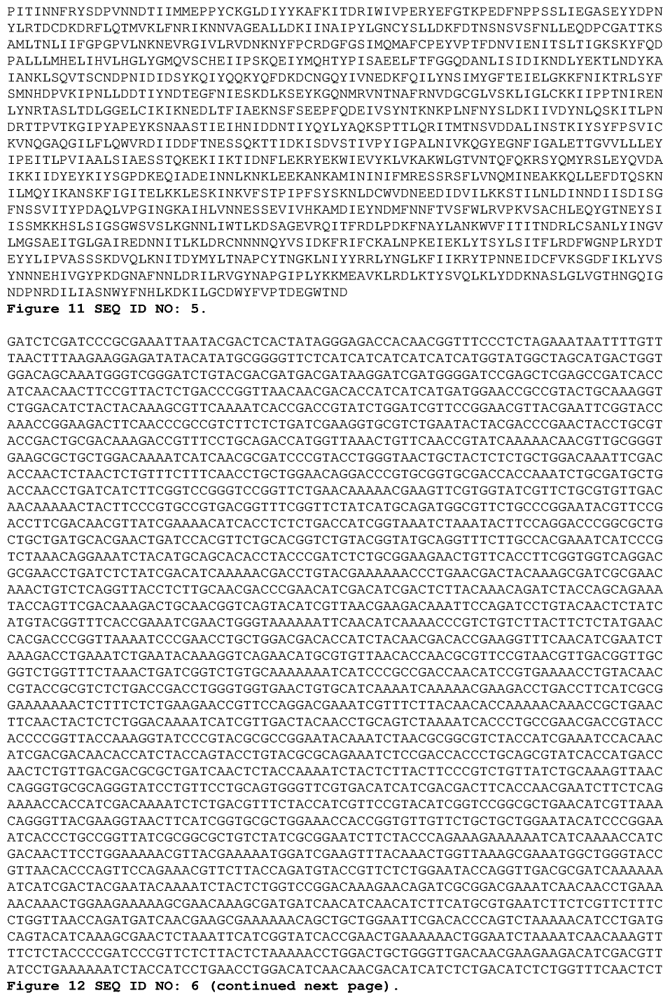

### Example 4—Preparation of PEG-HZN-TeNT-LC-HC (FIG. 11) (Method 1)

4.1—Preparation of TeNT

1. E. coli BL21 DE3 pLysS strain was electrotransformed with pRSET-TeNT vector and recovered in 1 mL LB media for 1 hour at 37′C.

2. 200 mL of pre-induction broth was inoculated with recovery culture. The pre-induction broth pH 7.2-7.4 comprised: 1.2% Tryptone; 2.4% Yeast extract; 2% Glucose; 0.4% Glycerol; 17 mM KH₂PO₄; 72 mM K HPO₄; and selection antibiotics (ampicillin and chloramphenicol).

3. The culture was incubated overnight at 30° C. with fast shaking.

4. The overnight culture was harvested by centrifugation at 4000 g for 10 minutes.

5. The pellet was resuspended in 200 mL expression broth pH 7.2-7.4, comprising: 1.2% Tryptone; 2.4% Yeast extract; 0.4% glycerol; 1 mM IPTG; 17 mM KH₂PO₄; 72 mM K₂HPO₄; 100 μg/mL ampicillin; and 10 μM ZnCl₂.

6. The protein was expressed for 6 hours at 30° C. with fast shaking.

7. The cells were harvested by centrifugation at 4500 g for 15 min and the pellet resuspended in 30 mL of TBS with 20 mM imidazole at pH 8.

8. The cells were lysed by sonication.

9. The cell lysate was cleared by centrifugation at 4500 g for 20 min and filtered through a 0.45 μm filter.

10. The protein was purified by His-tag affinity chromatography using the AKTA pure 25 FPLC system (GE).

11. The purified protein underwent buffer exchange to PBS using size exclusion chromatography followed by a second stage purification by gel filtration using AKTA pure 25 FPLC with Superdex 200 increase 10/300 GL column.

4.2—Endotoxin Removal

1. A 0.5 mL endotoxin removal spin column was equilibrated to room temperature.

2. The column bottom plug was removed, column cap loosened, column placed in a 15 mL tube then centrifuged at 500 g for 1 minute to remove solution from column. The solution was discarded.

3. The column bottom plug was replaced, column cap removed, 0.2N NaOH in 95% ethanol added to resin, column cap replaced, column inverted several times to resuspend resin, then incubated at room temperature for 1-2 h.

4. The column bottom plug was removed, column cap loosened, column place in a 15 mL tube then centrifuged at 500 g for 1 minute to remove solution from column. The solution was discarded.

5. The column bottom plug was replaced, column cap removed, endotoxin-free 2M NaCl added to resin, column cap replaced, and column inverted several times to resuspend resin.

6. The column bottom plug was removed, column cap loosened, column placed in a 15 mL tube then centrifuged at 500 g for 1 minute to remove solution from column. The solution was discarded.

7. The column bottom plug was replaced, column cap removed, endotoxin-free ultrapure water added to resin, column cap replaced, and column inverted several times to resuspend resin.

8. The column bottom plug was removed, column cap loosened, column placed in a 15 mL tube, and centrifuged at 500 g for 1 minute to remove solution from column. The solution was discarded.

9. The column bottom plug was replaced, column cap removed, endotoxin-free phosphate buffer added to resin, column cap replaced, and column inverted several times to resuspend resin.

10. The column bottom plug was removed, column cap loosened, column place in a 15 mL tube, and centrifuged at 500 g for 1 minute to remove solution from column. The solution was discarded.

11. The column was rinsed twice more with phosphate buffer and the eluate discarded.

12. The column bottom plug was replaced, column cap removed, sample applied to the resin, column cap replaced, and column inverted several times to resuspend resin.

13. The column was incubated with end-over-end mixing at 4° C. for at least 1 h.

14. The column bottom plug was removed, column cap loosened, column placed in an endotoxin-free 15 mL tube, and centrifuged at 500 g for 1 minute to remove solution from column. The sample was retained.

15. The endotoxin removal procedure was repeated with a regenerated spin column until the endotoxin levels in the sample were at an equivalent or lower level so that all dosages would contain less than 5 EU units of endotoxin per kilogram of the subject.

4.3—Generation of mPEG-HZN-NHS

1. A molar excess of SANH or SHNH was combined with mPEG-Propionaldehyde, with PEG of about 2 kDa, about 5 kDa, about 10 kDa or about 20 kDa, in PBS (pH6.5-7.5) or 10-100% DMF, under normal atmosphere, a nitrogen atmosphere or an argon atmosphere.

2. Attachment was performed between 4° C. and 37° C. for between 1 and 16 hours.

4.4—Preparation of PEG-HZN-TeNT-LC-HC

1. In a total volume of 500 pL PBS pH 7 or 10% DMF 3 μmol purified TeNT and 0.5 mmol mpeg-HZN-NHSwere combined with one of

2 kDa, 5 kDa, 10 kDa, 20 kDa, or 30 kDa PEG.

2. The sample was mixed at room temperature for 3 hours.

3. Excess PEG was removed by size exclusion chromatography.

4.5—Trypsin Digestion of Protein into Active Form

1. 1 mg of the protein was dissolved in 0.5 mL digestion buffer, comprising 0.1 M NH₄HCO₃ buffer, pH 8.0 or 0.1 M Tris buffer pH 8.5.

2. 0.10 mL to 0.25 mL of Immobilized TPCK Trypsin was washed with 3×500 μL of digestion buffer. The gel was separated from the buffer after each wash by centrifugation.

3. The gel was resuspended in about 0.2 mL of the digestion buffer.

4. The Immobilized TPCK Trypsin was added to the protein sample.

5. The reaction mixture was incubated for 2 hours to 18 hours at 37° C. in a rapidly shaking incubator.

6. The Immobilized TPCK Trypsin was separated by centrifugation.

### Example 5—Preparation of PEG-HZN-TeNT-LC-HC (FIG. 1I) (Method 2)

In this example, surface serine residues of TeNT-LC-HC were mutated to surface cysteine residues (S to C mutant) to facilitate directed PEG conjugation at immunogenic epitopes. The TeNT mutations were: S81C; S120C; S144C; S248C; S335C; S428C; S600C; S963C; S1041C; S1155C; and S1187C, relative to SEQ ID NO: 1.

5.1—Preparation of Serine Mutant TeNT-LC-HC

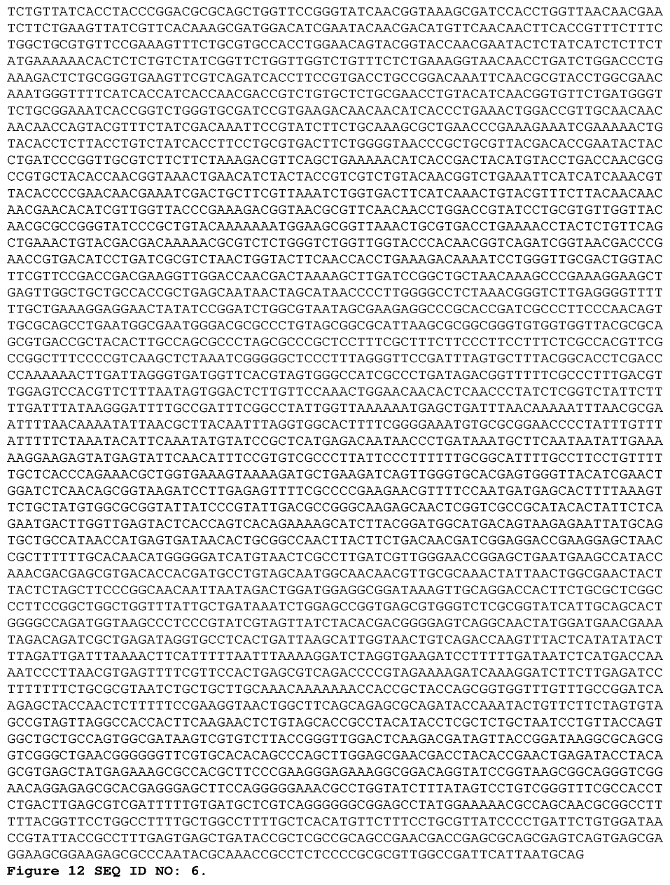

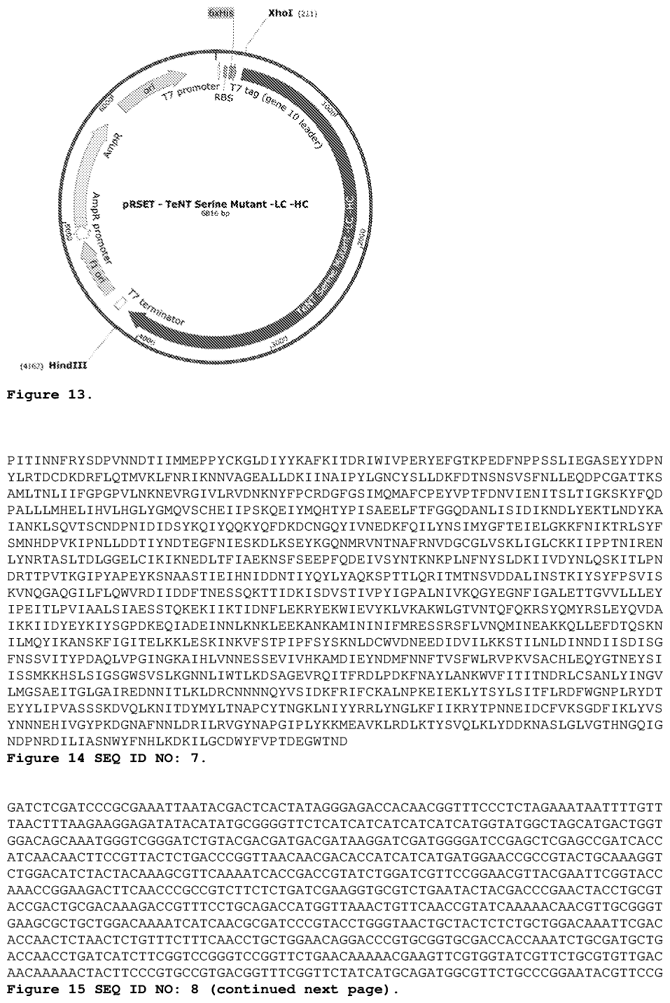

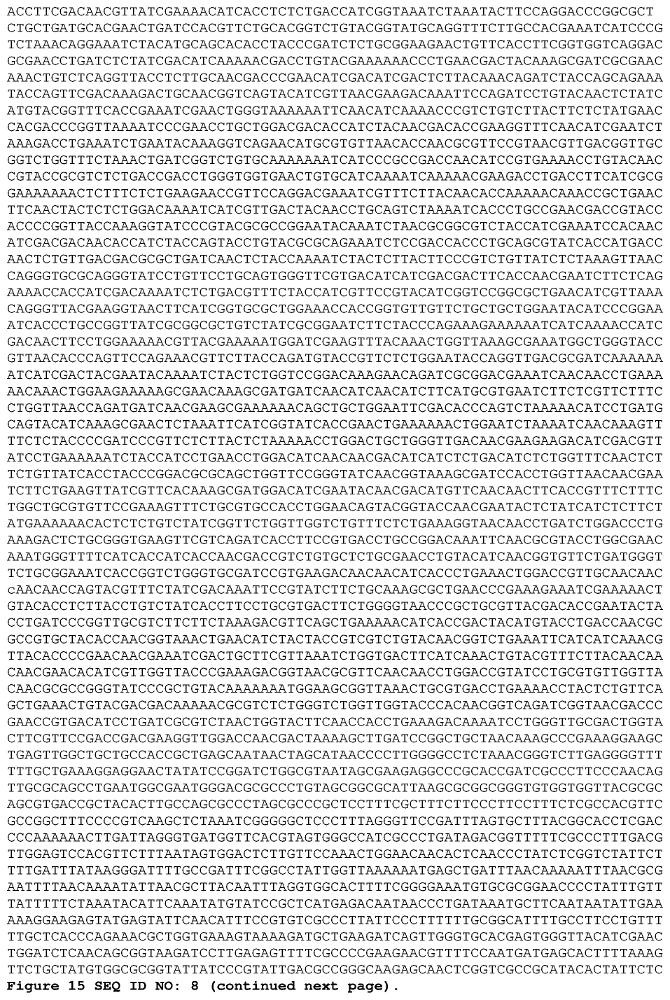

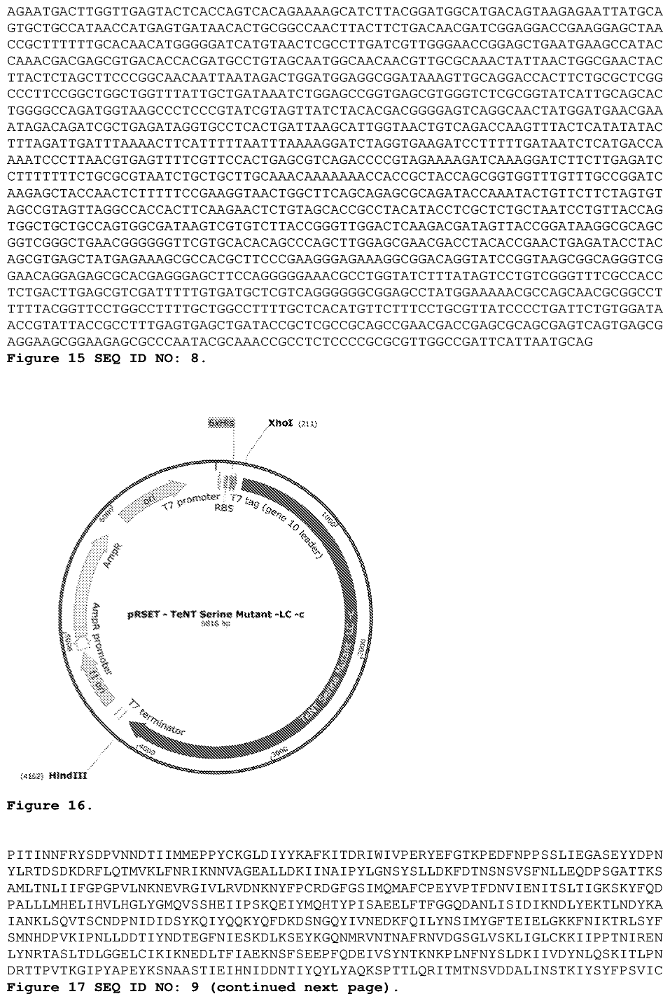

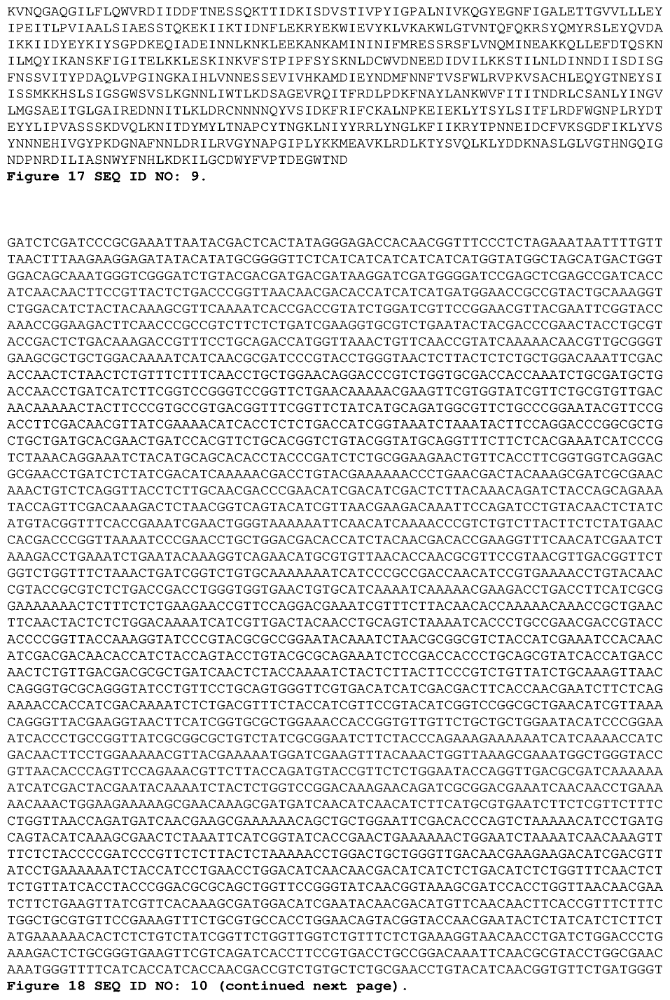

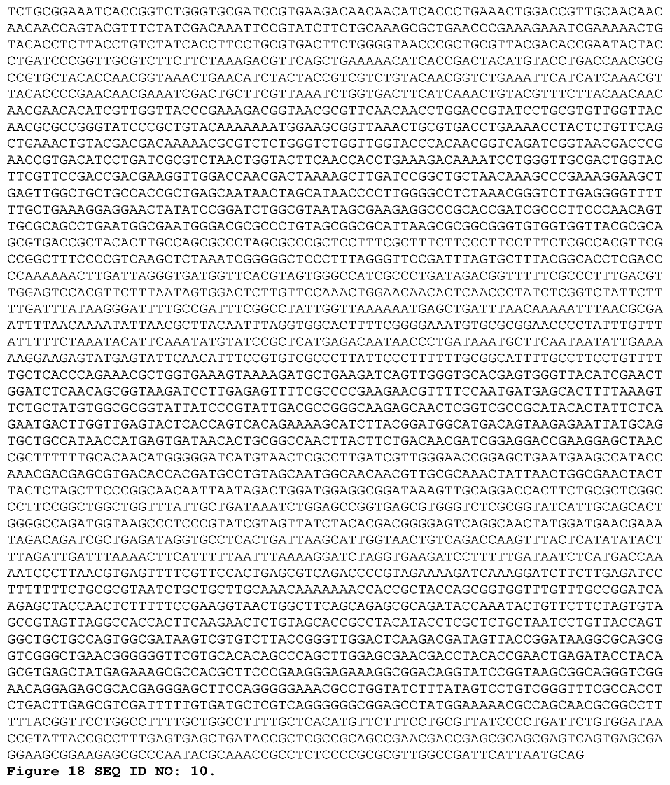

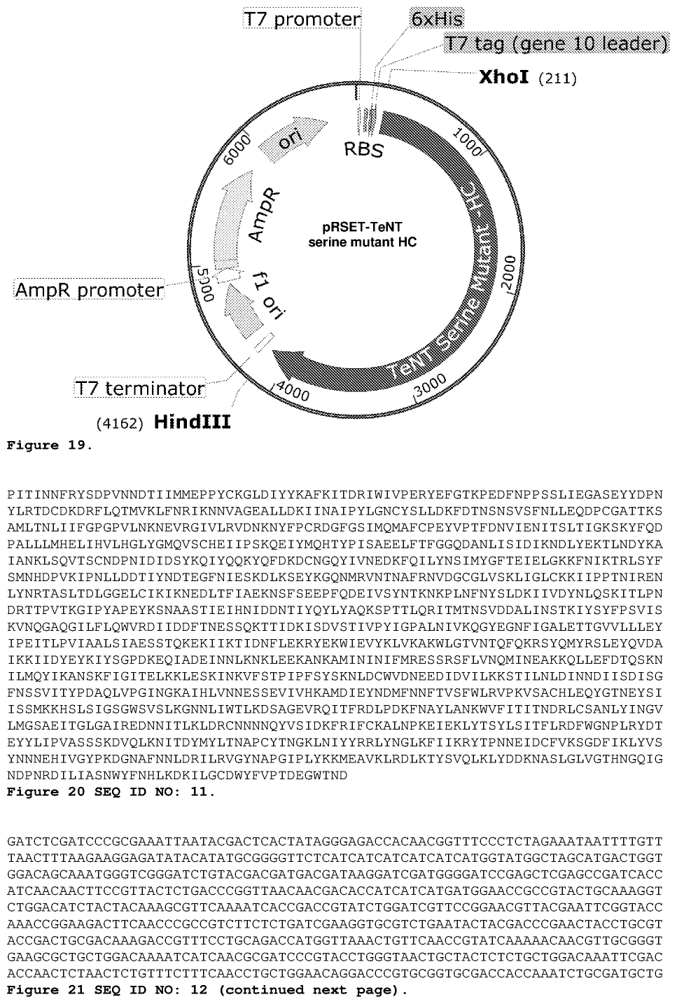

E. coli BL21 (DE3) pLysS strain was electrotransformed with vector pRSET-TeNT SC (FIGS. 12 and 13) encoding the amino acid sequence of FIG. 11 (SEQ ID NO: 6) comprising the S to C mutations. TeNT-LC-HC comprising the S to C mutations was expressed and purified according to Example 4 with the addition of treatment by 0.5 mM DTT for 15 minutes between step 10 and 11.

5.2—Endotoxin Removal

Endotoxin was removed from the TeNT-LC-HC Serine mutant according to Example 4.

5.3—PEG Attachment to Cysteine Residues

1. A molar excess of PEG-HZN-maleimide, with PEG of about 2 kDa, about 5 kDa, about 10 kDa or about 20 kDa, will be combined with serine mutant TeNT-LC-HC (0.5-2 mg/mL) in PBS at pH 6.5-7.5.

2. Attachment will be performed at room temperature for 2-16 hours.

3. Excess PEG will be removed by size exclusion chromatography.

5.4—Trypsin Digest Activation of Protein

Trypsin digestion for activation of the TeNT-LC-HC Serine mutant will be performed according to Example 4.

### Example 6 Synthesis of mPEG-HZN-NHS

In this example, methoxy-polyethylene glycol of various sizes (eg. 2 kDa, 5 kDa, 10 kDa, 20 kDa or 30 kDa) was attached to a sulfo-NHS group via a pH-labile hydrazone linkage. mPEG-propionaldehyde was combined with a molar excess of SANH or SHNH cross-linker in PBS, DMF or DMSO. The attachment was performed for 2-16 hours at room temperature under normal atmosphere, a nitrogen atmosphere or an argon atmosphere. Unreacted cross-linker may be removed by incubation with 1/10 moles of benzyloxybenzylaldehyde beads for 4-8 hours at room temperature.

### Example 7 Synthesis of mPEG-HZN-Maleimide

In this example, methoxy-polyethylene glycol of various sizes (eg. 2 kDa, 5 kDa, 10 kDa, 20 kDa or 30 kDa) was attached to a maleimide group via a pH-labile hydrazone linkage. mPEG-propionaldehyde was combined with an n-molar excess of BMPH (N-β-maleimidopropionic acid hydrazide) EMCH or KMUH cross-linker in DMF, DMSO or PBS. The attachment was performed for 1-16 hours at room temperature under a normal atmosphere, a nitrogen atmosphere or an argon atmosphere.

### Example 8—Preparation of PEP-TeNT-c (FIG. 1F)

In this example, surface serine residues of TeNT-c will be mutated to surface cysteine residues to facilitate directed GT-PEP conjugation at immunogenic epitopes to produce the molecule of FIG. 1F. The mutations will be: S963C, S1041C, S1155C, and S1187C.

The gene for TeNT with surface serine to cysteine substitutions S963C, S1041C, S1155C, and S1187C will be synthesised by a commercial provider, for example Integrated DNA Technologies. The gene will be sub-cloned into the pRSET-A expression vector by restriction digestion so that 6× Histidine tag from the vector is added to the N-terminus of the mutant protein.

TeNT comprising S963C, S1041C, S1155C, and S1187C will be expressed, GT-PEPylated and purified according to Example 11 to produce PEP-TeNT-c.

### Example 9—Preparation of PEP-TeNT-HC (FIG. 1B)

In this example, surface serine residues of TeNT-HC will be mutated to surface cysteine residues to facilitate directed PEG conjugation at immunogenic epitopes to produce the molecule of FIG. 1B. The mutations will be: S600C, S963C, S1041C, S1155C, and S1187C (FIG. 24 SEQ ID NO: 16).

The gene for TeNT with surface serine to cysteine substitutions S600C, S963C, S1041C, S1155C, and S1187C will be synthesised by a commercial provider, for example Integrated DNA Technologies. The gene will be sub-cloned into the pRSET-A expression vector (FIG. 25 SEQ ID NO: 17, FIG. 26) by restriction digestion so that 6× Histidine tag from the vector is added to the N-terminus of the mutant protein.

TeNT comprising S600C, S963C, S1041C, S1155C, and S1187C will be expressed, GT-PEPylated and purified according to Example 11 to produce PEP-TeNT-HC.

### Example 10—Preparation of PEP-TeNT-LC-c (FIG. 1C)

In this example, surface serine residues of LC and c will be mutated to surface cysteine residues to facilitate directed PEG conjugation at immunogenic epitopes to produce the molecule of FIG. 1C. The mutations were: S81C, S120C, S144C, S248C, S335C, S428C, S963C, S1041C, S1155C, and S1187C (FIG. 21 SEQ ID NO: 14).

The gene for TeNT with surface serine to cysteine substitutions S81C, S120C, S144C, S248C, S335C, S428C, S963C, S1041C, S1155C, and S1187C in LC and c will be synthesised by a commercial provider, Integrated DNA Technologies. The gene will be sub-cloned into the pRSET-A expression vector (FIG. 22 SEQ ID NO: 15, FIG. 23) by restriction digestion so that 6× Histidine tag from the vector was added to the N-terminus of the mutant protein.

TeNT comprising S81C, S120C, S144C, S248C, S335C, S428C, S963C, S1041C, S1155C, and S1187C will be expressed, GT-PEPylated and purified according to Example 11 to produce PEP-TeNT-LC-c.

10.1—Endotoxin Removal

Endotoxin will be removed from the TeNT-LC-c Serine mutant according to Example 4.

### Example 11—Preparation of PEP-TeNT-LC-RC (FIG. 1E) (method 1)

11.1—Preparation of TeNT

1. E. coli BL21 DE3 pLysS strain will be electrotransformed with pRSET-TeNT (SEQ ID 2 (FIG. 7). vector and recovered in 1 mL LB media for 1 hour at 37° C.

2. 200 mL of pre-induction broth will be inoculated with recovery culture. The pre-induction broth pH 7.2-7.4 comprised: 1.2% Tryptone; 2.4% Yeast extract; 2% Glucose; 0.4% Glycerol; 17 mM KH₂PO₄; 72 mM K₂HPO₄; and selection antibiotics (ampicillin and chloramphenicol).

3. The culture will be incubated overnight at 30° C. with fast shaking.

4. The overnight culture will be harvested by centrifugation at 4000 g for 10 minutes.

5. The pellet will be resuspended in 200 mL expression broth pH 7.2-7.4, comprising: 1.2% Tryptone; 2.4% Yeast extract; 0.4% glycerol; 1 mM IPTG; 17 mM KH₂PO₄; 72 mM K₂HPO₄; 100 μg/mL ampicillin; and 10 μM ZnCl₂.

6. The protein will be expressed for 6 hours at 30° C. with fast shaking.

7. The cells will be harvested by centrifugation at 4500 g for 15 min and the pellet resuspended in 30 mL of TBS with 20 mM imidazole at pH 8.

8. The cells will be lysed by sonication.

9. The cell lysate will be cleared by centrifugation at 4500 g for 20 min and filtered through a 0.45 μm filter.

10. The protein will be purified by His-tag affinity chromatography using the AKTA pure 25 FPLC system (GE).

11. The purified protein will undergo buffer exchange to PBS using size exclusion chromatography followed by a second stage purification by gel filtration using AKTA pure 25 FPLC with Superdex 200 increase 10/300 GL column.

11.2—Endotoxin Removal

1. A 0.5 mL endotoxin removal spin column will be equilibrated to room temperature.

2. The column bottom plug will be removed, column cap loosened, column placed in a 15 mL tube then centrifuged at 500 g for 1 minute to remove solution from column. The solution will be discarded.

3. The column bottom plug will be replaced, column cap removed, 0.2N NaOH in 95% ethanol added to resin, column cap replaced, column inverted several times to resuspend resin, then incubated at room temperature for 1-2 h.

4. The column bottom plug will be removed, column cap loosened, column place in a 15 mL tube then centrifuged at 500 g for 1 minute to remove solution from column. The solution will be discarded.

5. The column bottom plug will be replaced, column cap removed, endotoxin-free 2M NaCl added to resin, column cap replaced, and column inverted several times to resuspend resin.

6. The column bottom plug will be removed, column cap loosened, column placed in a 15 mL tube then centrifuged at 500 g for 1 minute to remove solution from column. The solution will be discarded.

7. The column bottom plug will be replaced, column cap removed, endotoxin-free ultrapure water added to resin, column cap replaced, and column inverted several times to resuspend resin.

8. The column bottom plug will be removed, column cap loosened, column placed in a 15 mL tube, and centrifuged at 500 g for 1 minute to remove solution from column. The solution will be discarded.

9. The column bottom plug will be replaced, column cap removed, endotoxin-free phosphate buffer added to resin, column cap replaced, and column inverted several times to resuspend resin.

10. The column bottom plug will be removed, column cap loosened, column place in a 15 mL tube, and centrifuged at 500 g for 1 minute to remove solution from column. The solution will be discarded.

11. The column will be rinsed twice more with phosphate buffer and the eluate discarded.

12. The column bottom plug will be replaced, column cap removed, sample applied to the resin, column cap replaced, and column inverted several times to resuspend resin.

13. The column will be incubated with end-over-end mixing at 4° C. for at least 1 h.

14. The column bottom plug will be removed, column cap loosened, column placed in an endotoxin-free 15 mL tube, and centrifuged at 500 g for 1 minute to remove solution from column. The sample will be retained.

15. The endotoxin removal procedure will be repeated with a regenerated spin column until the endotoxin levels in the sample were at an equivalent or lower level so that all dosages would contain less than 5 EU units of endotoxin per kilogram of the subject.

11.3—Method 1

In this example, GT-PEP-NHS will be attached to the primary amine groups on lysine residues on the surface of TeNT to produce the molecule from figure FIG. 1D.

1. A molar excess of GT-PEP-NHS, with PEP of about 2 kDa, about 5 kDa, about 10 kDa or about 20 kDa, will be combined with serine mutant TeNT-LC-c (0.5-2 mg/mL) in PBS at pH 6.5-7.5.

2. Attachment will be performed at room temperature for 6 hours.

3. Excess PEG will be removed by size exclusion chromatography.

11. 4—Trypsin Digest Activation of Protein

Trypsin digestion for activation of the PEP-TeNT-LC-HC Serine mutant will be performed according to Example 4.

11.5—Trypsin Digestion of Protein into Active Form

1. 1 mg of the protein will be dissolved in 0.5 mL digestion buffer, comprising 0.1 M NH₄HCO₃ buffer, pH 8.0 or 0.1 M Tris buffer pH 8.5.

2. 0.10 mL to 0.25 mL of Immobilized TPCK Trypsin will be washed with 3×500 μL of digestion buffer. The gel will be separated from the buffer after each wash by centrifugation.

3. The gel will be resuspended in about 0.2 mL of the digestion buffer.

4. The Immobilized TPCK Trypsin will be added to the protein sample.

5. The reaction mixture will be incubated for 2 hours to 18 hours at 37° C. in a rapidly shaking incubator.

6. The Immobilized TPCK Trypsin will be separated by centrifugation.

11.6—Method 2

In this example, surface serine residues of TeNT-LC-HC will be mutated to surface cysteine residues (S to C mutant) to facilitate directed GT-PEP conjugation at immunogenic epitopes and produce the molecule of figure FIG. 1D. The TeNT mutations were: S81C; S120C; S144C; S248C; S335C; S428C; S600C; S963C; S1041C; S1155C; and S1187C, relative to SEQ ID NO: 1.

1. A molar excess of GT-PEP-maleimide, with PEP of about 2 kDa, about 5 kDa, about 10 kDa or about 20 kDa, will be combined with serine mutant TeNT-LC-c (0.5-2 mg/mL) in PBS at pH 6.5-7.5.

2. Attachment will be performed at room temperature for 6 hours.

3. Excess PEG will be removed by size exclusion chromatography.

11.7—Trypsin Digest Activation of Protein

Trypsin digestion for activation of the TeNT-LC-c Serine mutant will be performed according to Example 4.

11.8—Trypsin Digestion of Protein into Active Form

1. 1 mg of the protein will be dissolved in 0.5 mL digestion buffer, comprising 0.1 M NH₄HCO₃ buffer, pH 8.0 or 0.1 M Tris buffer pH 8.5.

2. 0.10 mL to 0.25 mL of Immobilized TPCK Trypsin will be washed with 3×500 μL of digestion buffer. The gel will be separated from the buffer after each wash by centrifugation.

3. The gel will be resuspended in about 0.2 mL of the digestion buffer.

4. The Immobilized TPCK Trypsin will be added to the protein sample.

5. The reaction mixture will be incubated for 2 hours to 18 hours at 37° C. in a rapidly shaking incubator.

6. The Immobilized TPCK Trypsin will be separated by centrifugation.

### Example 12—Synthesis of GT-PEP-NHS

1. The gene for GT-PEPtide of approximately 2 kDa to 30 kDa with will be synthesised by a commercial provider, Integrated DNA Technologies. The gene will be sub-cloned into two expression vectors by restriction digestion so that a 6× Histidine tag from the vector will or will not be added to the N-terminus of the mutant protein.

2. E. coli BL21 DE3 pLysS strain will be electrotransformed with GT-PEPtide vector and recovered in 1 mL LB media for 1 hour at 37©C.

3. 200 mL of pre-induction broth will be inoculated with recovery culture. The pre-induction broth pH 7.2-7.4 will comprise: 1.2% Tryptone; 2.4% Yeast extract; 2% Glucose; 0.4% Glycerol; 17 mM KH₂PO₄; 72 mM K HPO₄; and selection antibiotics (ampicillin and chloramphenicol).

4. The culture will be incubated overnight at 30° C. with fast shaking.

5. The overnight culture will be harvested by centrifugation at 4000 g for 10 minutes.

6. The pellet will be resuspended in 200 mL expression broth pH 7.2-7.4, comprising: 1.2% Tryptone; 2.4% Yeast extract; 0.4% glycerol; 1 mM IPTG; 17 mM KH₂PO₄; 72 mM KHPO₄; 100 μg/mL ampicillin.

6. The protein will be expressed for 6 hours at 30° C. with fast shaking.

7. The cells will be harvested by centrifugation at 4500 g for 15 min and the pellet resuspended in 30 mL of TBS with 20 mM imidazole at pH 8.

8. The cells will be lysed by sonication.

9. The cell lysate will be cleared by centrifugation at 4500 g for 20 min and filtered through a 0.45 μm filter.

10. The protein will be purified by His-tag affinity chromatography using the AKTA pure 25 FPLC system (GE). Or by ammonium sulfate precipitation.

11. The purified protein will undergo buffer exchange and concentration to PBS by size-exclusion chromatography.

12. Sulfo NHS ester will be attached to the carboxyl terminus of the GT-PEPtides by incubation with a molar excess of EDC (1-Ethyl-3-(3-dimethylaminopropyl)carbodiimide) and Sulfo-NHS (N-Hydroxysulfosuccinimide) in PBS at room temperature for 2 to 8 hours.

13. Excess EDC and Sulfo HNS will be removed by dialysis, desalting or buffer exchange.

### Example 13—Synthesis of GT-PEP-Maleimide

1. The gene for GT-PEPtide of approximately 2 kDa to 30 kDa with will be synthesised by a commercial provider, Integrated DNA Technologies. The gene will sub-cloned into two expression vectors by restriction digestion so that a 6× Histidine tag from the vector will or will not be added to the N-terminus of the mutant protein.

2. E. coli BL21 DE3 pLysS strain will be electrotransformed with GT-PEPtide vector and recovered in 1 mL LB media for 1 hour at 37° C.

3. 200 mL of pre-induction broth will be inoculated with recovery culture. The pre-induction broth pH 7.2-7.4 will comprise: 1.2% Tryptone; 2.4% Yeast extract; 2% Glucose; 0.4% Glycerol; 17 mM KH₂PO₄; 72 mM K₂HPO₄; and selection antibiotics (ampicillin and chloramphenicol).

4. The culture will be incubated overnight at 30° C. with fast shaking.

5. The overnight culture will be harvested by centrifugation at 4000 g for 10 minutes.

6. The pellet will be resuspended in 200 mL expression broth pH 7.2-7.4, comprising: 1.2% Tryptone; 2.4% Yeast extract; 0.4% glycerol; 1 mM IPTG; 17 mM KH₂PO₄; 72 mM K₂HPO₄; 100 μg/mL ampicillin.

6. The protein will be expressed for 6 hours at 30° C. with fast shaking.

7. The cells will be harvested by centrifugation at 4500 g for 15 min and the pellet resuspended in 30 mL of TBS with 20 mM imidazole at pH 8.

8. The cells will be lysed by sonication.

9. The cell lysate will be cleared by centrifugation at 4500 g for 20 min and filtered through a 0.45 μm filter.

10. The protein will be purified by His-tag affinity chromatography using the AKTA pure 25 FPLC system (GE). Or by ammonium sulfate precipitation.

11. The purified protein will undergo buffer exchange and concentration to PBS by size-exclusion chromatography.

12. Maleimide groups will be attached to the amino terminus of the GT-PEPtides by incubation with a molar excess of Sulfo-SMCC (sulfosuccinimidyl 4-(N-maleimidomethyl)cyclohexane-1-carboxylate) in PBS at room temperature for 2 to 8 hours.

13. Excess Sulfo-SMCC will be removed by dialysis, desalting or buffer exchange.

### Example 14 Synthesis of GlycoGT-HEN-PEP-NRS

14.1 Generation of GlycoGT Peptides

1. Genes encoding repeating or random sequences of Glycine and threonine will be synthisized commercially and cloned into either a yeast or mammalian expression vector.

2. Peptides will be expressed in a glycosylating strain of yeast, such as sacharomyces cerivisie or a mammalian cell line, such as chinese hamspter ovaries.

3. Peptides will be purified by chromatographic methods such as anion exchange and size exlusion.

14.2 Addition of Aldehyde Groups to Glyco-Peptides

To ensure aldehyde groups are present on the carbohydrate residues, the GT-glycopeptides will be oxidised by Glycine or Glutathione.

14.3 Synthesis of Gly-HEN-NRS

1. GlycoGT-PEP will be attached to NHS ester via a pH-labile hydrazone linkage.

2. GlycoGT-PEP will be combined with an n-molar excess of SHNH or SANH cross-linker in DMF, DMSO or PBS.

3. The attachment will be performed for 1-16 hours at room temperature under a normal atmosphere, a nitrogen atmosphere or an argon atmosphere.

### Example 15—Synthesis of GlycoGT-HEN-PEP-Maleimide

15.1 Generation of GlycoGT Peptides

1. Genes encoding repeating or random sequences of Glycine and threonine will be synthisized commercially and cloned into either a yeast or mammalian expression vector.

2. Peptides will be expressed in a glycosylating strain of yeast, such as sacharomyces cerivisie or a mammalian cell line, such as chinese hamspter ovaries.

3. Peptides will be purified by chromatographic methods such as anion exchange and size exlusion.

15.2 Addition of aldehyde groups to Glyco-Peptides

To ensure aldehyde groups are present on the carbohydrate residues, the GT-glycopeptides will be oxidised by Glycine or Glutathione.

15.3 Synthesis of Gly-HEN-maleimide 1. GlycoGT-PEP will be attached to a maleimide group via a pH-labile hydrazone linkage.

2. GlycoGT-PEP will be combined with an n-molar excess of BMPH (N-β-maleimidopropionic acid hydrazide) EMCH (3,3′-N-[ε-Maleimidocaproic acid] hydrazide) or KMUH (N-κ-maleimidoundecanoic acid hydrazide) cross-linker in DMF, DMSO or PBS.

3. The attachment will be performed for 1-16 hours at room temperature under a normal atmosphere, a nitrogen atmosphere or an argon atmosphere.

### Example 16—PEG-HEN-TeNT Analysis

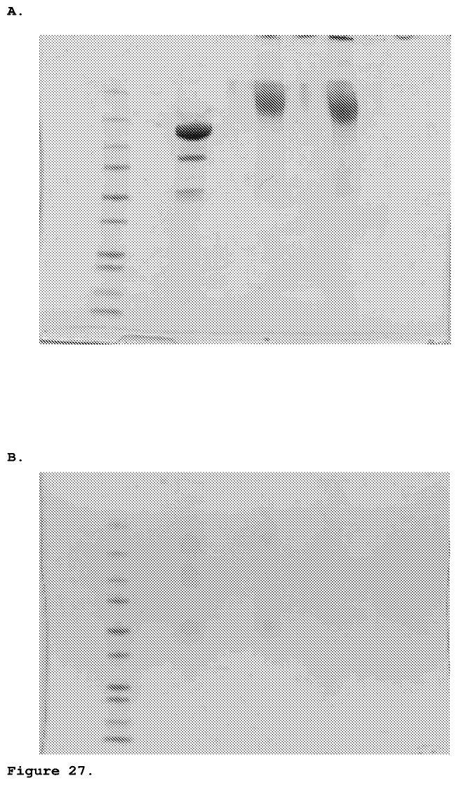

TeNT was prepared and PEGylated according to Example 4, then analysed by SDS-PAGE (FIG. 27) and detected (A) using Coomassie blue and (B) by Western blot using polyclonal anti-TeNT antibodies. FIG. 27(B) shows that antibody binding affinity is proportional to PEG molecular weight.

### Example 17—Reduced Immunogenicity of PEG-HEN-TeNT Versus TeNT

Four PEG-HZN-TeNTs each comprising a different molecular weight PEG (2 kDa, 5 kDa, 10 kDa and 20 kDa) will be prepared and PEGylated according to Example 4. The PEG-TeNTs were then assayed by competitive ELISA against TeNT.

In the first assay (FIG. 29A), TeNT will be adsorbed to an ELISA plate. Adsorbed TeNT will then be probed with a polyclonal anti-TeNT antibody pre-incubated with each of four concentrations (10 μg/mL, 1 μg/mL, 0.1 μg/mL and 0.01 μg/mL) of four PEG-TeNT antigens (2 kDa, 5 kDa, 10 kDa and 20 kDa). In this assay, higher responses (OD 450 nm) indicated greater affinity for TeNT and therefore reduced immunogenicity for the PEG-TeNT.

In the second assay (FIG. 29B), each PEG-TeNT will be adsorbed to a separate ELISA plate. Each adsorbed PEG-TeNT (2 kDa, 5 kDa, 10 kDa and 20 kDa) will then be probed with a polyclonal anti-TeNT antibody pre-incubated with each of four concentrations (10 μg/mL, 1 μg/mL, 0.1 μg/mL and 0.01 μg/mL) of TeNT antigen. In this assay, lower responses (OD 450 nm) indicated greater affinity for TeNT and therefore reduced immunogenicity for the PEG-TeNT.

This example will show that anti-TeNT antibodies bind preferentially to TeNT and that PEGylated TeNTs have reduced immunogenicity relative to TeNT (i.e. non-PEGylated).

### Example 18—Reduced Imunogenicity of PEG-HZN-TeNT-LC-c Serine Mutant Versus TeNT-LC-c Serine Mutant

TeNT-LC-c Serine mutant will be prepared according to Example 4. Four samples of the TeNT-LC-c Serine mutant were PEGylated according to Example 4, each sample comprising a different molecular weight PEG (2 kDa, 5 kDa, 10 kDa and 20 kDa). The PEG-HZN-TeNT-LC-c Serine mutants will be assayed by competitive ELISA against TeNT-LC-c Serine mutant.

In the first assay, TeNT-LC-c Serine mutant will be adsorbed to an ELISA plate. Adsorbed TeNT Serine mutant will be then probed with a polyclonal anti-TeNT antibody pre-incubated with each of four concentrations (10 μg/mL, 1 μg/mL, 0.1 μg/mL and 0.01 μg/mL) of four PEG-HZN-TeNT-LC-c Serine mutant antigens (2 kDa, 5 kDa, 10 kDa and 20 kDa). In this assay, higher responses (OD 450 nm) indicated greater affinity for TeNT-LC-c Serine mutant and therefore reduced immunogenicity for the PEG-HZN-TeNT-LC-c Serine mutant.

In the second assay, each PEG-HZN-TeNT-LC-c Serine mutant will be adsorbed to a separate ELISA plate. Each adsorbed PEG-HZN-TeNT-LC-c Serine mutant (2 kDa, 5 kDa, 10 kDa and 20 kDa) will be then probed with a polyclonal anti-TeNT antibody pre-incubated with each of four concentrations (10 μg/mL, 1 μg/mL, 0.1 μg/mL and 0.01 μg/mL) of TeNT-LC-c Serine mutant antigen. In this assay, lower responses (OD 450 nm) indicated greater affinity for TeNT-LC-c Serine mutant and therefore reduced immunogenicity for the PEG-HZN-TeNT-LC-c Serine mutant.

This example will show that anti-TeNT antibodies bind preferentially to TeNT-LC-c Serine mutant and that PEG-HZNTeNT-LC-c Serine mutants have reduced immunogenicity relative to TeNT-LC-c Serine mutant (i.e. non-PEGylated).

### Example 19—Reduced Immunogenicity of PEG-HEN-TeNT Versus TeNT

A competitive ELISA assay will be conducted in accordance with Example 9, except that the polyclonal antibody will be replaced by human serum collected from one or more subjects who have received a booster tetanus toxoid vaccination within the previous 12 months. Antibodies in the serum will show greater affinity for TeNT (i.e. non-PEGylated TeNT) versus PEG-HZN-TeNTs.

### Example 20—In Vivo Model

PEG-HZN-TeNT-LC-HC will be prepared by attaching PEG (about 2 kDa, 5 kDa, about 10 kDa or about 20 kDa) to the surface exposed lysine residues of recombinant TeNT according to Example 4.

One or more units of PEG-HZN-TeNT-LC-HC in 15 μL of PBS will be injected into the hind limb of female C57BL/6 mice. Each animal exhibited localised limb tetany within 48 hours of injection.

### Example 21—In Vivo Model

PEP-TeNT-LC-HC will be prepared by attaching GT PEPtide (about 2 kDa, 5 kDa, about 10 kDa or about 20 kDa) to the surface exposed lysine or cysteine residues of recombinant TeNT or recombinant TeNT Serine mutant according to Example 11.

One or more units of PEP-TeNT-LC-HC in 15 μL of PBS will be injected into the hind limb of female C57BL/6 mice. Each animal will exhibit localised limb tetany within 48 hours of injection

### Example 22

PEG-HZN-TeNT-LC-HC comprising 2 kDa, 5 kDa, 10 kDa or 20 kDa PEG will be administered at 50-500 000 ng/kg intramuscularly to the hind leg muscle of mice previously immunized with tetanus toxoid. Increased muscle contraction will be observed in the injected muscle for up to 3 days, and will be greater than the effect observed in mice administered the same units of TeNT.

### Example 23

PEP-TeNT-LC-HC comprising 2 kDa, 5 kDa, 10 kDa or 20 kDa PEP will be administered at 50-500 000 ng/kg intramuscularly to the hind leg muscle of mice previously immunized with tetanus toxoid. Increased muscle contraction will be observed in the injected muscle for up to 3 days, and will be greater than the effect observed in mice administered the same units of TeNT.

### Example 24

PEP-HZN-TeNT-LC-HC comprising 2 kDa, 5 kDa, 10 kDa or 20 kDa PEP will be administered at 50-500 000 ng/kg intramuscularly to the hind leg muscle of mice previously immunized with tetanus toxoid. Increased muscle contraction will be observed in the injected muscle for up to 3 days, and will be greater than the effect observed in mice administered the same units of TeNT or PEP-TeNT-LC-HC.

### Example 25

GlycoPEP-HZN-TeNT-LC-HC comprising 2 kDa, 5 kDa, 10 kDa or 20 kDa PEG will be administered at 50-500 000 ng/kg intramuscularly to the hind leg muscle of mice previously immunized with tetanus toxoid. Increased muscle contraction will be observed in the injected muscle for up to 3 days, and will be greater than the effect observed in mice administered the same units of TeNT or PEP-TeNT-LC-HC.

### Example 26

Bulldogs of approximately 30 kg will be administered 25-50 000 ng/kg PEG-HZN-TeNT-c comprising 20 kDa PEG intramuscularly with the dose divided bilaterally to the left and right geniohyoid. Upon administration, obstructive sleep apnoea (OSA) will decrease in PEG-HZN-TeNT treated animals compared with animals treated with vehicle alone. The bulldogs will be observed weekly for OSA and the PEG-HZN-TeNT-c dose will be repeated as needed until efficacy decreases, as determined by a return of OSA comparable to animals treated with vehicle alone.

Thereafter, the bulldogs will be administered 25-50 000 ng/kg of PEG-HZN-TeNT-HC or 25-50 000 ng/kg PEG-HZN-TeNT-LC-c, each comprising 20 kDa PEG, divided bilaterally to the left and right geniohyoid. Upon administration, OSA will decrease in PEG-HZN-TeNT treated animals compared with animals treated with vehicle alone. The bulldogs will be observed weekly for OSA and the PEG-HZN-TeNT-HC and PEG-HZN-TeNT-LC-c dose will be repeated as needed until efficacy decreases, as determined by a return of OSA comparable to animals treated with vehicle alone.

Thereafter, the bulldogs will be administered 25-50 000 ng/kg PEG-HZN-TeNT-LC-HC, comprising 20 kDa PEG, divided bilaterally to the left and right geniohyoid. Upon administration, OSA will decrease in PEG-HZN-TeNT treated animals compared with animals treated with vehicle alone. The bulldogs will be observed weekly for OSA and the PEG-HZN-TeNT-LC-HC dose will be repeated as needed until efficacy decreases, as determined by a return of OSA comparable to animals treated with vehicle alone.

### Example 27

Bulldogs of approximately 30 kg will be administered 25-50 000 ng/kg PEP-TeNT-c or PEP-HZN-TeNT-c comprising 20 kDa PEP intramuscularly with the dose divided bilaterally to the left and right geniohyoid. Upon administration, obstructive sleep apnoea (OSA) will decrease in PEP-TeNT or PEP-HZN-TeNT treated animals compared with animals treated with vehicle alone. The bulldogs will be observed weekly for OSA and the PEG-HZN-TeNT-c dose will be repeated as needed until efficacy decreases, as determined by a return of OSA comparable to animals treated with vehicle alone.

Thereafter, the bulldogs will be administered 25-50 000 ng/kg of PEP-TeNT-HC or PEP-HZN-TeNT-HC or 25-50 000 ng/kg PEP-TeNT-LC-c or PEP-HZN-TeNT-LC-c, each comprising 20 kDa PEP, divided bilaterally to the left and right geniohyoid. Upon administration, OSA will decrease in PEP-TeNT or PEP-HZN-TeNT treated animals compared with animals treated with vehicle alone. The bulldogs will be observed weekly for OSA and the PEP-TeNT-HC or PEP-HZN-TeNT-HC and PEP-TeNT-LC-c or PEP-HZN-TeNT-LC-c dose will be repeated as needed until efficacy decreases, as determined by a return of OSA comparable to animals treated with vehicle alone.

Thereafter, the bulldogs will be administered 25-50 000 ng/kg PEP-TeNT-LC-HC or PEP-HZN-TeNT-LC-HC, comprising 20 kDa PEP, divided bilaterally to the left and right geniohyoid. Upon administration, OSA will decrease in PEP-TeNT or PEP-HZN-TeNT treated animals compared with animals treated with vehicle alone. The bulldogs will be observed weekly for OSA and the PEP-TeNT-LC-HC or PEP-HZN-TeNT-LC-HC dose will be repeated as needed until efficacy decreases, as determined by a return of OSA comparable to animals treated with vehicle alone.

1. A tetanus neurotoxin (TeNT) conjugate comprising a TeNT conjugated to at least one masking moiety:
(a) through an acid labile linkage; or
(b) the masking moiety comprising a masking polypeptide.

2. The tetanus neurotoxin conjugate of claim 1, wherein the masking moiety comprises a PEG and/or a masking polypeptide.

3. (canceled)

4. The tetanus neurotoxin conjugate of claim 1 conjugated to at least one masking moiety through an acid labile linkage.

5. The tetanus neurotoxin conjugate of claim 1, wherein the masking moiety is conjugated to a lysine or a cysteine residue of the TeNT.

6. The tetanus neurotoxin conjugate of claim 5, wherein the cysteine is an introduced amino acid.

7. The tetanus neurotoxin conjugate of claim 1, wherein the tetanus neurotoxin comprises the amino acid sequence represented by SEQ ID NO: 1.

8. The tetanus neurotoxin conjugate of claim 7, wherein the introduced cysteine is a serine to cysteine amino acid substitution relative to SEQ ID NO: 1.

9. The tetanus neurotoxin conjugate of claim 1, wherein the acid labile linkage comprises hydrazone.

10. A composition comprising the tetanus neurotoxin conjugate of claim 1.

11. A method of treating hypotonia in a subject, comprising administering a tetanus neurotoxin conjugate of claim 1.

12. A method of enhancing muscle power and/or muscle tone and/or muscle healing and/or sporting performance in a subject, comprising administering the tetanus neurotoxin conjugate of claim 1.
